(19) United States
(12) Patent Application Publication
(10) Pub. No.: US 2022/0071970 A1
(43) Pub. Date: Mar. 10, 2022

(54) QUINUCLIDINE-3-ONE DERIVATIVES AND THEIR USE IN CANCER TREATMENT

(71) Applicant: Aprea Therapeutics AB, Solna (SE)

(72) Inventors: Lars Hagberg, Stockholm (SE); Rune Ringom, Stockholm (SE); Tim Blizzard, Stockholm (SE)

(21) Appl. No.: 17/276,431

(22) PCT Filed: Sep. 20, 2019
(86) PCT No.: PCT/EP2019/075278

§ 371 Date: Mar. 15, 2021

(30) Foreign Application Priority Data
Sep. 20, 2018 (EP) 18195621.0
Sep. 24, 2018 (EP) 18196157.4
Sep. 26, 2018 (EP) 18196777.9
Sep. 28, 2018 (EP) 18197436.1

(51) Int. Cl. A61K 31/439; A61K 51/10; A61P 35/00; C07D 453/02; C07D 519/00; A61K 31/444; A61K 31/506

(52) U.S. Cl. CPC A61K 31/439; A61K 51/10; A61P 35/00; A61K 31/506; C07D 519/00; A61K 31/444; C07D 453/02

(57) ABSTRACT

The invention relates to certain substituted quinuclidine-3-one compounds for use in the treatment of hyperproliferative disease, such as cancer, and diseases associated with inflammation. More particularly, the present invention relates to certain substituted 3-quinuclidinones, pharmaceutically acceptable salts thereof, pharmaceutical compositions containing the same, and to methods for using such compounds. In this manner, these compounds are of use for treating hyperproliferative diseases and inflammatory diseases.

### FIELD OF THE INVENTION

The invention relates to certain substituted quinuclidine-3-one compounds for use in the treatment of hyperproliferative disease, such as cancer, and diseases associated with inflammation. More particularly, the present invention relates to certain substituted 3-quinuclidinones, pharmaceutically acceptable salts thereof, pharmaceutical compositions containing the same, and to methods for using such compounds. In this manner, these compounds are of use for treating hyperproliferative diseases and inflammatory diseases.

### BACKGROUND OF THE INVENTION

The fact that around half of all human tumors carry mutations in TP53, the gene encoding p53, is solid testimony as to the critical role of this protein as tumor suppressor. p53 halts the cell cycle and/or triggers apoptosis in response to various stress stimuli, including DNA damage, hypoxia, and oncogene activation (Ko & Prives (1996), Genes Dev 10:1054-1072; Sherr (1998), Genes Dev 12:2984-2991). Upon activation, p53 initiates the p53-dependent biological responses through transcriptional transactivation of specific target genes carrying p53 DNA binding motifs. In addition, the multifaceted p53 protein may promote apoptosis through transactivation-independent effects; in the nucleus repression of certain genes, and in the cytoplasmic space involving sequestering the anti-apoptotic protein Bcl-xL (Bennett et al (1998), Science 282:290-293; Gottlieb & Oren (1998), Semin Cancer Biol 8:359-68; Ko & Prives (1996), supra; Green et al (2009), Nature 458:1127-1130). Analyses of a large number of mutant TP53 genes in human tumors have revealed a strong selection for mutations that inactivate the specific DNA binding function of the resulting “mutant” p53; most TP53 mutations in tumors are point mutations clustered in the part encoding the DNA binding core domain of p53 (residues 94-292) (Beroud & Soussi (1998), Nuci Acids Res 26:200-204). The p53 core domain also binds the anti-apoptotic Bcl-xL, involving a surface partially overlapping the DNA binding surface.

Both p53-induced cell cycle arrest and apoptosis could be involved in p53-mediated tumor suppression. While p53-induced cell cycle arrest could conceivably be reversed in different ways, p53-induced cell death would have the advantage of being irreversible. There is indeed evidence from animal in vivo models (Symonds et al (1994), Cell 78:703-711) and human tumors (Bardeesy et al (1995), Cancer Res 55:215-219) indicating that p53-dependent apoptosis plays a major role in the elimination of emerging tumors, particularly in response to oncogenic signaling. Moreover, the ability of p53 to induce apoptosis often determines the efficacy of cancer therapy (Lowe et al (1994), Science 266:807-810). Taking into account the fact that more than 50% of human tumors carry p53 mutations, it appears highly desirable to restore the function of wild type p53-mediated apoptosis to tumors. The advantage of this approach is that it will allow selective elimination of tumor cells carrying mutant p53, since these are particularly sensitive to p53 reactivation, supposedly for two main reasons. Firstly, tumor cells are sensitized to apoptosis due to oncogene activation (reviewed in Evan & Littlewood (1998), Science 281:1317-1322). Secondly, mutant p53 proteins tend to accumulate at high levels in tumor cells. Therefore, restoration of the wild type function to the abundant and presumably “activated” mutant p53 should trigger a massive apoptotic response in already sensitized tumor cells, whereas normal cells that harbor low or undetectable levels of p53 should not be affected. The feasibility of p53 reactivation as an anticancer strategy is supported by recent data on quinuclidine-3-one derivatives, suggesting that a therapeutic strategy based on rescuing p53-induced apoptosis may be widely applicable (Bykov et al (2016), Front Oncol 6, article 21).

It may be that malfunctioning of the p53 pathway is generally involved in a number of diseases, such as those enumerated herein above. Indeed, in addition to hyperproliferative diseases such as cancer, various authors have shown the involvement of deficient p53 functioning in a number of other disease states, e.g. autoimmune diseases and cardiac diseases.

Thus, Mountz et al (1994), Arthritis and Rheumatology 10:1415-1420, state that human autoimmune diseases share the common feature of an imbalance between the production and destruction of various cell types including lymphocytes (SLE), synovial cells (Ra), and fibroblasts (scleroderma). Proto-oncogenes which regulate apoptosis, including bcl-2, TP53, and myc, are also expressed abnormally. According to the authors, specific therapies that induce apoptosis without incurring side effects should improve treatment of autoimmune disease.

Okuda et al (2003), J Neuroimmunol 135:29-37, present results suggesting that p53 may be involved in the regulatory process of experimental autoimmune encephalomyelitis (EAE) through the control of cytokine production and/or the apoptotic elimination of inflammatory cells. EAE as a model for autoimmune inflammatory diseases of the central nervous system (CNS) is a widely used model for the human disease multiple sclerosis.

In addition to stabilizing the folded conformation of mutant p53, treatment with a 2,2-substituted quinuclidine-3-one has been shown to result in reversible inhibition of thioredoxin reductase (TrxR) 1, and also to deplete the cells of glutathione (Peng et al (2013), Cell Death Dis 4:e881; Mohell et al (2015), Cell Death Dis 6:e1794; Liu et al (2017), Nat Commun 8:14844). Hence, a 2,2-substituted quinuclidine-3-one derivative leads to suppression of both branches of the cellular defense against oxidative stress, which has been shown to have an anti-cancer effect (Wondrak (2009), Antioxid Redox Signal 11:3015-3069). The redox effects of 2,2-substituted quinuclidine-3-one derivatives suggest that this type of compounds may have a beneficial effect in chronic inflammatory diseases, comprising allergy, asthma, atherosclerosis, coeliac disease, Crohn's disease, gout, inflammatory bowel disease, rheumatoid arthritis, and transplant rejection.

A number of quinuclidine-3-one derivatives that are able to induce apoptosis of cells carrying mutant p53 are set forth in WO2002/24692, WO2003/070250, WO2004/084893 and WO2005/090341. WO2004/084893, for example, generally describes quinuclidine-3-one derivatives that are capable of inducing apoptosis in malignant melanoma cells, such as amide-containing quinuclidine-3-one derivatives. However, no examples of amide-containing quinuclidine-3-one derivatives are disclosed. Nonetheless, there still remains a general need of compounds having activity in the treatment of disorders and diseases related to p53 malfunctioning and/or oxidative stress. Preferably, such compounds should have improved pharmacokinetic and pharmacodynamic properties. Preferably, such compounds should have improved physicochemical properties. One main objective of the present invention is to provide such compounds.

WO2015/150472 describes a method of treating melanoma using a 2,2-substituted quinuclidine-3-one in a combination therapy with a BRAF inhibitor. The quinuclidine-3-one derivatives generally described in WO2015/150472 may be substituted with an optionally further substituted monocyclic heteroaromatic ring. However, no examples of compounds bearing such a substituted or unsubstituted monocyclic heteroaromatic ring are disclosed.

WO2007/062030 and CN104860994 describe a series of 2,2-substituted quinuclidine-3-one derivatives for the treatment of cancer by restoring the activity of mutant p53.

The use of quinuclidine-3-one derivatives for inducing apoptosis via p53 in breast cancer cells is described by Malki et al (2017), Bioorg Chem 72:57-63.

Certain 2-substituted 3-quinuclidinones have been described earlier in a biological context, but not in the therapeutic areas mentioned above. Thus, 2-[N′—(O-alkoxyphenyl)piperazinomethyl]-3-quinuclidinones (U.S. Pat. No. 3,598,825) have been described as nervous system depressants. Amine-substituted 2-methylene 3-quinuclidinones have been described as anti-bacterial agents (U.S. Pat. No. 3,726,877) and antidepressants (U.S. Pat. No. 3,462,442). U.S. Pat. No. 3,384,641 describes a method wherein 2-methylene-3-quinuclidinone is reacted with amines to form intermediates which, upon heating, could release the amines. The intermediates thus obtained are used for the purification of amines.

### DESCRIPTION OF THE INVENTION

The present invention provides certain novel compounds, pharmaceutically acceptable salts, hydrates, solvates and combinations thereof and pharmaceutical compositions containing the same, as well as methods and uses for treating disease. The quinuclidine-3-one derivatives of the present invention are useful in the treatment of hyperproliferative diseases, autoimmune diseases, inflammatory diseases and heart diseases. In particular, they are useful in the treatment of disorders involving malfunctioning of the p53 pathway.

The compounds of the present invention have advantageous properties relating to the ability to kill tumor cells, including apoptosis of tumor cells carrying mutant p53. The compounds also display favorable pharmacokinetic and pharmacodynamic properties, a high potency, stability in formulation, low toxicity, and show synergistic effects with other anti-cancer agents.

In the most general form, the present invention provides a compound of formula (I)

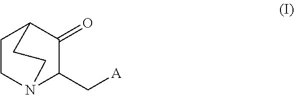

wherein

A represents

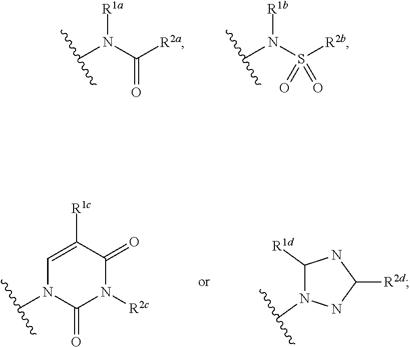

R^{1a} is selected from the group consisting of H, C₁-C₆ alkyl, C₁-C₆ haloalkyl, C₃-C₆ cycloalkyl and C₃-C₆ cyclohaloalkyl, said alkyl, haloalkyl, cycloalkyl and cyclohaloalkyl being optionally substituted with one or more C₁-C₆ alkoxy;

R^{2a} is C₁-C₆ haloalkyl;

R^{1b} is selected from the group consisting of H, C₁-C₆ alkyl, C₁-C₆ haloalkyl, C₃-C₆ cycloalkyl, C₃-C₆ cyclohaloalkyl, phenyl, halogenated phenyl, benzyl, halogenated benzyl and —CH₂—R^{3b}, said alkyl, haloalkyl, cycloalkyl, cyclohaloalkyl, phenyl and halogenated phenyl being optionally substituted with one or more C₁-C₃ alkyl, C₁-C₃ haloalkyl, C₁-C₆ alkoxy or C₁-C₆ haloalkoxy;

R^{2b} is selected from the group consisting of C₁-C₆ alkyl, C₁-C₆ haloalkyl, C₃-C₆ cycloalkyl, C₃-C₆ cyclohaloalkyl, phenyl, halogenated phenyl, benzyl, halogenated benzyl, heteroaryl and halogenated heteroaryl, said alkyl, haloalkyl, cycloalkyl, cyclohaloalkyl, phenyl, halogenated phenyl, benzyl, halogenated benzyl, heteroaryl and halogenated heteroaryl being optionally substituted with one or more C₁-C₃ alkyl, C₁-C₃ haloalkyl, C₁-C₆ alkoxy or C₁-C₆ haloalkoxy;

R^{3b} is selected from the group consisting of heterocyclyl, COOR^{4b} and CONR^{5b}R^{6b};

R^{4b} is selected from the group consisting of H, C₁-C₆ alkyl and C₁-C₆ haloalkyl;

R^{5b} and R^{6b} are the same or different and are selected from the group consisting of H, C₁-C₆ alkyl and C₁-C₆ haloalkyl;

R^{1c} is selected from the group consisting of H, C₁-C₆ alkyl, C₁-C₆ haloalkyl, C₃-C₆ cycloalkyl and C₃-C₆ cyclohaloalkyl, said alkyl, haloalkyl, cycloalkyl and cyclohaloalkyl being optionally substituted with one or more C₁-C₆ alkoxy;

R^{2c} is selected from the group consisting of H, —CH₂—R^{3c} and —COOR^{4c};

R^{3c} is heterocyclyl;

R^{4c} is selected from the group consisting of C₁-C₆ alkyl and C₁-C₆ haloalkyl;

R^{1d} is selected from the group consisting of H, C₁-C₆ alkyl, C₁-C₆ haloalkyl, C₃-C₆ cycloalkyl and C₃-C₆ cyclohaloalkyl, said alkyl, haloalkyl, cycloalkyl and cyclohaloalkyl being optionally substituted with one or more C₁-C₆ alkoxy or halogen; and

R^{2d} is selected from the group consisting of H, halogen, cyano,—COOR^{3d} and —CONR^{4d}R^{5d};

R^{3d} is selected from the group consisting of H, C₁-C₆ alkyl and C₁-C₆ haloalkyl; and

R^{4d} and R^{5d} are the same or different and selected from the group consisting of H, C₁-C₆ alkyl and C₁-C₆ haloalkyl;

or an enantiomer, mixture of enantiomers, pharmaceutically acceptable salt, hydrate, solvate or combination thereof.

In the most general form, the present invention also provides a pharmaceutical composition comprising said compound of formula (I) or an enantiomer, mixture of enantiomers, pharmaceutically acceptable salt, hydrate, solvate or combination thereof.

Further, the present invention provides a compound of formula (I) or an enantiomer, mixture of enantiomers, pharmaceutically acceptable salt, hydrate, solvate or combination thereof, or a pharmaceutical composition, for use in the treatment of a disease associated with a malfunctioning p53 signaling pathway, for example associated with mutant p53.

Further, the present invention provides a method of treating a disease associated with a malfunctioning p53 signaling pathway, for example associated with mutant p53, comprising administering a compound of formula (I) or an enantiomer, mixture of enantiomers, pharmaceutically acceptable salt, hydrate, solvate or combination thereof, or a pharmaceutical composition comprising said compound of formula (I) or an enantiomer, mixture of enantiomers, pharmaceutically acceptable salt, hydrate, solvate or combination thereof, to a subject in need thereof.

Further, the present invention provides a method of preparing a compound of formula (I).

Further, the present invention provides use of a compound of formula (I), or an enantiomer, mixture of enantiomers, pharmaceutically acceptable salt, solvate, hydrate or combination thereof, in preparing a medicament for treating a disease associated with a malfunctioning p53 signaling pathway, for example associated with mutant p53

According to a first aspect of a first configuration, the present invention provides a compound of formula (II)

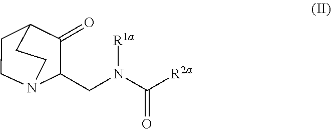

wherein

R^{1a} is selected from the group consisting of H, C₁-C₆ alkyl, C₁-C₆ haloalkyl, C₃-C₆ cycloalkyl and C₃-C₆ cyclohaloalkyl, said alkyl, haloalkyl, cycloalkyl and cyclohaloalkyl being optionally substituted with one or more C₁-C₆ alkoxy; and

R^{2a} is C₁-C₆ haloalkyl;

or an enantiomer, mixture of enantiomers, pharmaceutically acceptable salt, hydrate, solvate or combination thereof.

According to one embodiment of formula (II), R^{1a} is selected from the group consisting of H, C₁-C₆ alkyl and C₁-C₆ haloalkyl, said alkyl and haloalkyl being optionally substituted with one or more C₁-C₆ alkoxy; and R^{2a} is C₁-C₆ haloalkyl.

According to one embodiment of formula (II), R^{1a} is selected from the group consisting of H and C₁-C₆ alkyl, said alkyl being optionally substituted with one or more C₁-C₆ alkoxy; and R^{2a} is C₁-C₆ haloalkyl.

According to one embodiment of formula (II), R^{1a} is selected from the group consisting of H and ethyl; and R^{2a} is C₁-C₆ haloalkyl.

According to one embodiment of formula (II), R^{1a} is selected from the group consisting of H and ethyl; and R^{2a} is selected from the group consisting of trihalomethyl and dihalomethyl.

According to one embodiment of formula (II), R^{1a} is selected from the group consisting of H and ethyl; and R^{2a} is trihalomethyl.

According to one embodiment of formula (II), R^{1a} is selected from the group consisting of H and ethyl; and R^{2a} is selected from the group consisting of CHF₂, CF₃ and CCl₃.

According to one embodiment of formula (II), R^{1a} is selected from the group consisting of H and ethyl; and R² is selected from the group consisting of CF₃ and CCl₃.

According to one embodiment of formula (II), R^{1a} is H.

According to one embodiment of formula (II), R^{1a} is C₁-C₆ alkyl.

According to one embodiment of formula (II), R^{1a} is ethyl.

According to one embodiment of formula (II), R^{2a} is C₁-C₆ haloalkyl.

According to one embodiment of formula (II), R^{2a} is trihalomethyl.

According to one embodiment of formula (II), R^{2a} is selected from the group consisting of CF₃ and CCl₃.

According to one embodiment of formula (II), R^{2a} is dihalomethyl.

According to one embodiment of formula (II), R^{2a} is CHF₂.

According to one embodiment of this aspect of the present invention, there is provided a compound selected from the group consisting of:
2,2,2-trichloro-N-ethyl-N-((3-oxoquinuclidin-2-yl)methyl)acetamide;
2,2,2-trichloro-N-((3-oxoquinuclidin-2-yl)methyl)acetamide;
N-ethyl-2,2,2-trifluoro-N-((3-oxoquinucl idin-2-yl)methyl)acetamide;
2,2,2-trifluoro-N-((3-oxoquinuclidin-2-yl)methyl)acetamide; and
2,2-difluoro-N-((3-oxoquinuclidin-2-yl)methyl)acetamide,

or an enantiomer, mixture of enantiomers, pharmaceutically acceptable salt, hydrate, solvate or combination thereof.

According to one embodiment of this aspect of the present invention, there is provided a compound selected from the group consisting of:
2,2,2-trichloro-N-ethyl-N-((3-oxoquinuclidin-2-yl)methyl)acetamide;
2,2,2-trichloro-N-((3-oxoquinuclidin-2-yl)methyl)acetamide;
N-ethyl-2,2,2-trifluoro-N-((3-oxoquinuclidin-2-yl)methyl)acetamide; and
2,2,2-trifluoro-N-((3-oxoquinuclidin-2-yl)methyl)acetamide,

or an enantiomer, mixture of enantiomers, pharmaceutically acceptable salt, hydrate, solvate or combination thereof

According to a second aspect of the first configuration, the present invention provides a pharmaceutical composition comprising said compound of formula (II) or an enantiomer, mixture of enantiomers, pharmaceutically acceptable salt, hydrate, solvate or combination thereof.

According to a third aspect of the first configuration, the present invention provides a compound of formula (II) or an enantiomer, mixture of enantiomers, pharmaceutically acceptable salt, hydrate, solvate or combination thereof according to the first aspect of the first configuration, or a pharmaceutical composition according to the second aspect of the first configuration, for use in the treatment of a disease associated with a malfunctioning p53 signaling pathway, for example associated with mutant p53.

According to a related, fourth aspect of the first configuration, the present invention provides a method of treating a disease associated with a malfunctioning p53 signaling pathway, for example associated with mutant p53, comprising administering a compound of formula (II) or an enantiomer, mixture of enantiomers, pharmaceutically acceptable salt, hydrate, solvate or combination thereof according to the first aspect of the first configuration, or a pharmaceutical composition according to the second aspect of the first configuration, to a subject in need thereof.

According to a fifth aspect of the first configuration, the present invention provides a method of preparing a compound according to the first aspect of the first configuration.

Examples of the synthesis of certain compounds of formula (II) are represented in the following reaction scheme:

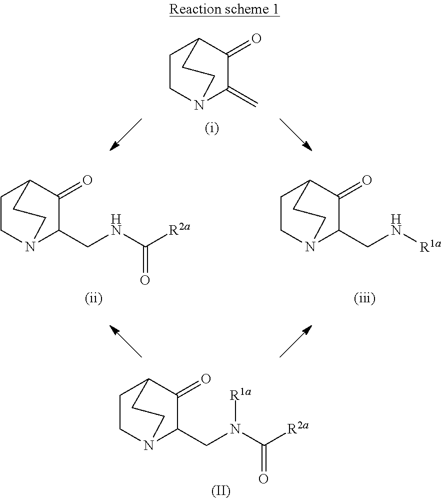

According to the Reaction scheme 1, 2-methylene-3-quinuclidinone (i) can be used as the starting material for the synthesis of the compounds (ii), (iii) and (11).

Compound (ii) can be made by reacting an amide with 2-methylene-3-quinuclidinone in the presence of an appropriate base according to the examples below.

Compound (iii) can be made by reacting 2-methylene-3-quinuclidinone with an amine in organic solvents as described by Malki et al (2017), supra; Singh et al (1969), J Med Chem 12:524-526 and U.S. Pat. No. 3,726,877, or in a mixture of an organic solvent and water in the presence of a phase transfer catalyst as described in WO2005/090341.

Compound (II) can be made from compound (iii) by reaction with an acyl chloride in the presence of a base according to the examples below.

The synthesis of compound (II) from compound (ii) may be performed by methods well known to the person skilled in the art by reacting it with an alkyl halide and a base in an organic solvent as described in WO2010/090976. Alternatively, an alcohol may be used to alkylate the nitrogen under Mitsunobu conditions as described by Orain & Mattes (2005), Synlett 19:3008-3010.

The synthesis of compound (II) from compound (i) may be performed by methods well known to the person skilled in the art by reacting it with a secondary amide and a base in an organic solvent as described by Sani et al (2017), Chemistry—A Eur J 23:5842-5850.

According to a first aspect of a second configuration, the present invention provides a compound of formula (III)

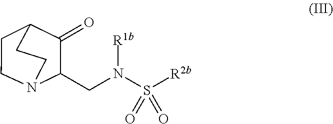

wherein

R^{1b} is selected from the group consisting of H, C₁-C₆ alkyl, C₁-C₆ haloalkyl, C₃-C₆ cycloalkyl, C₃-C₆ cyclohaloalkyl, phenyl, halogenated phenyl, benzyl, halogenated benzyl and —CH₂—R^{3b}, said alkyl, haloalkyl, cycloalkyl, cyclohaloalkyl, phenyl and halogenated phenyl being optionally substituted with one or more C₁-C₃ alkyl, C₁-C₃ haloalkyl, C₁-C₆ alkoxy or C₁-C₆ haloalkoxy;

R^{2b} is selected from the group consisting of C₁-C₆ alkyl, C₁-C₆ haloalkyl, C₃-C₆ cycloalkyl, C₃-C₆ cyclohaloalkyl, phenyl, halogenated phenyl, benzyl, halogenated benzyl, heteroaryl and halogenated heteroaryl, said alkyl, haloalkyl, cycloalkyl, cyclohaloalkyl, phenyl, halogenated phenyl, benzyl, halogenated benzyl, heteroaryl and halogenated heteroaryl being optionally substituted with one or more C₁-C₃ alkyl, C₁-C₃ haloalkyl, C₁-C₆ alkoxy or C₁-C₆ haloalkoxy;

R^{3b} is selected from the group consisting of heterocyclyl, COOR^{4b} and CONR^{5b}R^{6b};

R^{4b} is selected from the group consisting of H, C₁-C₆ alkyl and C₁-C₆ haloalkyl; and

R^{5b} and R^{6b} are the same or different and are selected from the group consisting of H, C₁-C₆ alkyl and C₁-C₆ haloalkyl;

or an enantiomer, mixture of enantiomers, pharmaceutically acceptable salt, hydrate, solvate or combination thereof.

According to one embodiment of formula (III), R^{1b} is selected from the group consisting of H, C₁-C₆ alkyl, C₃-C₆ cycloalkyl, phenyl and —CH₂—R^{3b}, said alkyl, cycloalkyl and phenyl being optionally substituted with one or more C₁-C₆ alkoxy or C₁-C₆ haloalkoxy.

According to one embodiment of formula (III), R^{1b} is selected from the group consisting of H, C₁-C₆ alkyl, C₃-C₆ cycloalkyl, phenyl and —CH₂—R^{3b}.

According to one embodiment of formula (III), R^{1b} is selected from the group consisting of H, ethyl, —CH₂-(3-oxoquinuclidin-2-yl), CH₂CONH₂, CH₂CO₂H, cyclopropyl and phenyl.

According to one embodiment of formula (III), R^{2b} is selected from the group consisting of C₁-C₆ alkyl, C₁-C₆ haloalkyl, C₃-C₆ cycloalkyl, phenyl, halogenated phenyl and heteroaryl, said alkyl, cycloalkyl, phenyl, and heteroaryl being optionally substituted with one or more C₁-C₃ alkyl, C₁-C₃ haloalkyl, C₁-C₆ alkoxy or C₁-C₆ haloalkoxy.

According to one embodiment of formula (III), R^{2b} is selected from the group consisting of C₁-C₆ alkyl, C₁-C₆ haloalkyl, C₃-C₆ cycloalkyl, phenyl, halogenated phenyl and heteroaryl.

According to one embodiment of formula (III), R^{2b} is selected from the group consisting of C₁-C₆ alkyl, C₁-C₆ haloalkyl, C₃-C₆ cycloalkyl, phenyl, halogenated phenyl and pyridinyl.

According to one embodiment of formula (III), R^{2b} is selected from the group consisting of C₁-C₆ alkyl, C₁-C₆ haloalkyl, C₃-C₆ cycloalkyl, phenyl, 4-fluorphenyl, 2-pyridinyl, 3-pyridinyl and 4-pyridinyl.

According to one embodiment of formula (III), R^{2b} is selected from the group consisting of methyl, trifluoromethyl, isopropyl, cyclopropyl, 1-methylcyclopropyl, phenyl, 4-fluorphenyl, 2-pyridinyl, 3-pyridinyl and 4-pyridinyl.

According to one embodiment of formula (III), R^{3b} is selected from the group consisting of 3-oxoquinuclidin-2-yl, COOR^{4b} and CONR^{5b}R^{6b}.

According to one embodiment of formula (III), R^{4b} is H.

According to one embodiment of formula (III), R^{5b} and R^{6b} are both H.

According to one embodiment of this aspect of the second configuration of the present invention, there is provided a compound selected from the group consisting of:
N-((3-oxoquinuclidin-2-yl)methyl)pyridine-3-sulfonamide;
4-fluoro-N-((3-oxoquinuclidin-2-yl)methyl)benzenesulfonamide;
N-ethyl-N-((3-oxoquinuclidin-2-yl)methyl)methanesulfonamide;
N-((3-oxoquinuclidin-2-yl)methyl)methanesulfonamide;
N-((3-oxoquinuclidin-2-yl)methyl)benzenesulfonamide;
2-(N-((3-oxoquinuclidin-2-yl)methyl)methylsulfonamido)acetamide;
N-(methylsulfonyl)-N-((3-oxoquinuclidin-2-yl)methyl)glycine;
N-((3-oxoquinuclidin-2-yl)methyl)pyridine-4-sulfonamide;
N-((3-oxoquinuclidin-2-yl)methyl)pyridine-2-sulfonamide;
N-ethyl-1,1,1-trifluoro-N-((3-oxoquinuclidin-2-yl)-methyl)methanesulfonamide;
1,1,1-trifluoro-N-((3-oxoquinuclidin-2-yl)methyl)methanesulfonamide;
N,N-bis((3-oxoquinuclidin-2-yl)methyl)methanesulfonamide;
N-((3-oxoquinuclidin-2-yl)methyl)propane-2-sulfonamide;
N-((3-oxoquinuclidin-2-yl)methyl)cyclopropanesulfonamide;
1-methyl-N-((3-oxoquinuclidin-2-yl)methyl)cyclopropane-1-sulfonamide;
N-cyclopropyl-N-((3-oxoquinuclidin-2-yl)methyl)methanesulfonamide; and
N-((3-oxoquinuclidin-2-yl)methyl)-N-phenylmethanesulfonamide;

or an enantiomer, mixture of enantiomers, pharmaceutically acceptable salt, hydrate, solvate or combination thereof.

According to a second aspect of the second configuration, the present invention provides a pharmaceutical composition comprising said compound of formula (III) or an enantiomer, mixture of enantiomers, pharmaceutically acceptable salt, hydrate, solvate or combination thereof.

According to a third aspect of the second configuration, the present invention provides a compound of formula (III) or an enantiomer, mixture of enantiomers, pharmaceutically acceptable salt, hydrate, solvate or combination thereof according to the first aspect of the second configuration, or a pharmaceutical composition according to the second aspect of the second configuration, for use in the treatment of a disease associated with a malfunctioning p53 signaling pathway, for example associated with mutant p53.

According to a related, fourth aspect of the second configuration, the present invention provides a method of treating a disease associated with a malfunctioning p53 signaling pathway, for example associated with mutant p53, comprising administering a compound of formula (III) or an enantiomer, mixture of enantiomers, pharmaceutically acceptable salt, hydrate, solvate or combination thereof according to the first aspect of the second configuration, or a pharmaceutical composition according to the second aspect of the second configuration, to a subject in need thereof.

According to a fifth aspect of the second configuration, the present invention provides a method of preparing a compound according to the first aspect of the second configuration.

Examples of the synthesis of certain compounds of formula (III) are represented in the following Reaction scheme 2:

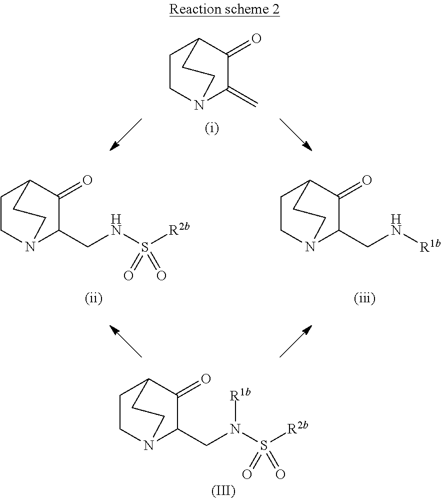

According to the Reaction scheme, 2-methylene-3-quinuclidinone (i) can be used as the starting material for the synthesis of the compounds (ii), (iii) and U.S. Pat. No. 3,726,877, or in a mixture of an organic solvent and water in the presence of a phase transfer catalyst as described in WO2005/090341.

Compound (III) can be made from compound (iii) by reaction with a sulfonyl chloride in the presence of a base according to the examples below.

The synthesis of compound (III) from compound (ii) may be performed by methods well known to the person skilled in the art by reacting it with an alkyl halide and a base in an organic solvent as described by Declerck et al (2004), J Org Chem 69:8372-8381. Alternatively, an alcohol may be used to alkylate the nitrogen under Mitsunobu conditions as described by Lee et al (2016), Org Lett 18:3678-3681.

The synthesis of compound (III) from compound (i) may be performed by methods well known to the person skilled in the art by reacting it with a secondary sulfonamide and a base in an organic solvent as described by Moriwake et al (1989), J Org Chem 54:4114-4120.

According to a first aspect of a third configuration, the present invention provides a compound of formula (IV)

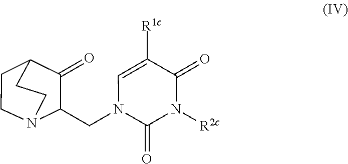

wherein

R^{1c} is selected from the group consisting of H, C₁-C₆ alkyl, C₁-C₆ haloalkyl, C₃-C₆ cycloalkyl and C₃-C₆ cyclohaloalkyl, said alkyl, haloalkyl, cycloalkyl and cyclohaloalkyl being optionally substituted with one or more C₁-C₆ alkoxy;

R^{2c} is selected from the group consisting of H, —CH₂—R^{3c} and —COOR^{4c};

R^{3c} is heterocyclyl; and

R^{4c} is selected from the group consisting of C₁-C₆ alkyl and C₁-C₆ haloalkyl;

or an enantiomer, mixture of enantiomers, pharmaceutically acceptable salt, hydrate, solvate or combination thereof.

According to one embodiment of formula (IV), R^{1c} is selected from the group consisting of H, C₁-C₆ alkyl and C₁-C₆ haloalkyl, said alkyl and haloalkyl being optionally substituted with one or more C₁-C₆ alkoxy;

R^{2c} is selected from the group consisting of H, —CH₂—R^{3c} and —COOR^{4c};

R^{3c} is heterocyclyl; and

R^{4c} is selected from the group consisting of C₁-C₆ alkyl and C₁-C₆ haloalkyl.

According to one embodiment of formula (IV), R^{1c} is selected from the group consisting of H and C₁-C₆ alkyl;

R^{2c} is selected from the group consisting of H, —CH₂—R^{3c} and —COOR^{4c};

R^{3c} is heterocyclyl; and

R^{4c} is selected from the group consisting of C₁-C₆ alkyl and C₁-C₆ haloalkyl.

According to one embodiment of formula (IV), R^{1c} is selected from the group consisting of H and methyl;

R^{2c} is selected from the group consisting of H, —CH₂—R^{3c} and —COOR^{4c};

R^{3c} is heterocyclyl; and

R^{4c} is tert-butyl.

According to one embodiment of formula (IV), R^{1c} is selected from the group consisting of H and methyl;

R^{2c} is selected from the group consisting of H, —CH₂—R^{3c} and —COOR^{4c};

R^{3c} is 3-oxoquinuclidin-2-yl; and

R^{4c} is tert-butyl.

According to one embodiment of formula (IV), R^{1c} is selected from the group consisting of H, C₁-C₆ alkyl, C₁-C₆ haloalkyl, C₃-C₆ cycloalkyl and C₃-C₆ cyclohaloalkyl, said alkyl, haloalkyl, cycloalkyl and cyclohaloalkyl being optionally substituted with one or more C₁-C₆ alkoxy;

R^{2c} is selected from the group consisting of H and —CH₂—R^{3c}; and

R^{3c} is heterocyclyl.

According to one embodiment of formula (IV), R^{1c} is selected from the group consisting of H, C₁-C₆ alkyl and C₁-C₆ haloalkyl, said alkyl and haloalkyl being optionally substituted with one or more C₁-C₆ alkoxy;

R^{2c} is selected from the group consisting of H and —CH₂—R^{3c}; and

R^{3c} is heterocyclyl.

According to one embodiment of formula (IV), R^{1c} is selected from the group consisting of H and C₁-C₆ alkyl;

R^{2c} is selected from the group consisting of H and —CH₂—R^{3c}; and

R^{3c} is heterocyclyl.

According to one embodiment of formula (IV), R^{1c} is selected from the group consisting of H and methyl;

R^{2c} is selected from the group consisting of H and —CH₂—R^{3c}; and

R^{3c} is heterocyclyl.

According to one embodiment of formula (IV), R^{1c} is selected from the group consisting of H and methyl;

R^{2c} is selected from the group consisting of H and —CH₂—R^{3c}; and

R^{3c} is 3-oxoquinuclidin-2-yl.

According to one embodiment of formula (IV), R^{1c} is H.

According to one embodiment of formula (IV), R^{1c} is C₁-C₆ alkyl.

According to one embodiment of formula (IV), R^{1c} is methyl.

According to one embodiment of formula (IV), R^{2c} is H.

According to one embodiment of formula (IV), R^{3c} is heterocyclyl.

According to one embodiment of formula (IV), R^{3c} is 3-oxoquinuclidin-2-yl.

According to one embodiment of formula (IV), R^{4c} is tert-butyl.

According to one embodiment of this aspect of the third configuration of the present invention, there is provided a compound selected from the group consisting of:
1-((3-oxoquinuclidin-2-yl)methyl)pyrimidine-2,4(1H,3H)-dione;
5-methyl-1-((3-oxoquinuclidin-2-yl)methyl)pyrimidine-2,4(1H,3H)-dione;
tert-butyl 5-methyl-2,6-dioxo-3-((3-oxoquinuclidin-2-yl)methyl)-3,6-dihydropyrimidine-1(2H)-carboxylate; and
5-methyl-1,3-bis((3-oxoquinuclidin-2-yl)methyl)pyrimidine-2,4(1H,3H)-dione;

or an enantiomer, mixture of enantiomers, pharmaceutically acceptable salt, hydrate, solvate or combination thereof.

According to one embodiment of this aspect of the third configuration of the present invention, there is provided a compound selected from the group consisting of:
1-((3-oxoquinuclidin-2-yl)methyl)pyrimidine-2,4(1H,3H)-dione;
5-methyl-1-((3-oxoquinuclidin-2-yl)methyl)pyrimidine-2,4(1H,3H)-dione; and
5-methyl-1,3-bis((3-oxoquinuclidin-2-yl)methyl)pyrimidine-2,4(1H,3H)-dione;

or an enantiomer, mixture of enantiomers, pharmaceutically acceptable salt, hydrate, solvate or combination thereof.

According to a second aspect of the third configuration, the present invention provides a pharmaceutical composition comprising said compound of formula (IV) or an enantiomer, mixture of enantiomers, pharmaceutically acceptable salt, hydrate, solvate or combination thereof.

According to a third aspect of the third configuration, the present invention provides a compound of formula (IV) or an enantiomer, mixture of enantiomers, pharmaceutically acceptable salt, hydrate, solvate or combination thereof according to the first aspect of the third configuration, or a pharmaceutical composition according to the second aspect of the third configuration, for use in the treatment of a disease associated with a malfunctioning p53 signaling pathway, for example associated with mutant p53.

According to a related, fourth aspect of the third configuration, the present invention provides a method of treating a disease associated with a malfunctioning p53 signaling pathway, for example associated with mutant p53, comprising administering a compound of formula (IV) or an enantiomer, mixture of enantiomers, pharmaceutically acceptable salt, hydrate, solvate or combination thereof according to the first aspect of the third configuration, or a pharmaceutical composition according to the second aspect of the third configuration, to a subject in need thereof.

According to a fifth aspect of the third configuration, the present invention provides a method of preparing a compound according to the first aspect of the third configuration.

Examples of the synthesis of certain compounds of formula (IV) are represented in the following Reaction scheme 3:

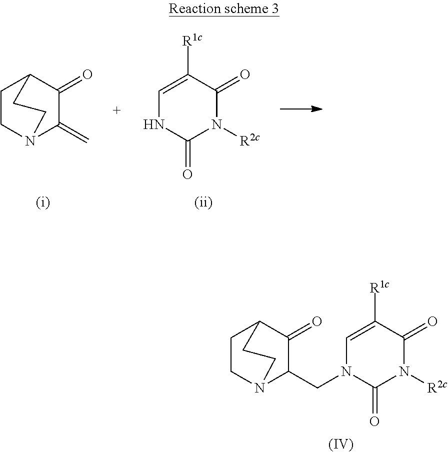

According to the Reaction scheme 3, compound (IV) can be made by reacting compound (ii) with 2-methylene-3-quinuclidinone (i) in the presence of an appropriate base in an organic solvent according to the examples below.

Compound (IV) (R^{2c}=(3-oxoquinuclidin-2-yl)methyl) can be made by reacting compound (ii) (R^{2c}═H) with 2-methylene-3-quinuclidinone (i), in appropriate stoichiometric amounts, in the presence of a base in an organic solvent according to the example below.

Compound (IV) may be made by reacting 2-methylene-3-quinuclidinone (i) with compound (ii) in DMF as described in WO2005/090341.

According to a first aspect of a fourth configuration, the present invention provides a compound of formula (V)

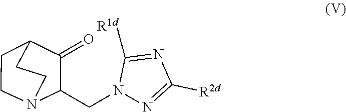

wherein

R^{1d} is selected from the group consisting of H, C₁-C₆ alkyl, C₁-C₆ haloalkyl, C₃-C₆ cycloalkyl and C₃-C₆ cyclohaloalkyl, said alkyl, haloalkyl, cycloalkyl and cyclohaloalkyl being optionally substituted with one or more C₁-C₆ alkoxy or halogen; and

R^{2d} is selected from the group consisting of H, halogen, cyano, —COOR^{3d} and —CONR^{4d}R^{5d};

R^{3d} is selected from the group consisting of H, C₁-C₆ alkyl and C₁-C₆ haloalkyl; and

R^{4d} and R^{5d} are the same or different and selected from the group consisting of H, C₁-C₆ alkyl and C₁-C₆ haloalkyl;

or an enantiomer, mixture of enantiomers, pharmaceutically acceptable salt, hydrate, solvate or combination thereof.

According to one embodiment of formula (V), R^{1d} is selected from the group consisting of H, C₁-C₆ alkyl and C₁-C₆ haloalkyl, said alkyl and haloalkyl being optionally substituted with one or more C₁-C₆ alkoxy; and R^{2d}, R^{3d}, R^{4d} and R^{5d} are as described above.

According to one embodiment of formula (V), R^{1d} is selected from the group consisting of H and C₁-C₆ alkyl, said alkyl being optionally substituted with one or more C₁-C₆ alkoxy; and R^{2d}, R^{3d}, R^{4d} and R^{5d} are as described above.

According to one embodiment of formula (V), R^{1d} is H; and R^{2d}, R^{3d}, R^{4d} and R^{5d} are as described above.

According to one embodiment of formula (V), R^{1d} is H; R^{2d} is selected from the group consisting of H, halogen, cyano and —CONR^{4d}R^{5d}; and R^{4d} and R^{5d} are as described above.

According to one embodiment of formula (V), R^{1d} is H; R^{2d} is selected from the group consisting of H, halogen, cyano and —CONR^{4d}R^{5d}; and R^{4d} and R^{5d} are the same or different and selected from the group consisting of H and C₁-C₆ alkyl.

According to one embodiment of formula (V), R^{1d} is H; R^{2d} is selected from the group consisting of H, chloride, cyano and —CONR^{4d}R^{5d}; and R^{4d} and R^{5d} are the same or different and selected from the group consisting of H and methyl.

According to one embodiment of formula (V), R^{1d} is H.

According to one embodiment of formula (V), R^{2d} is selected from H, chloride and cyano.

According to one embodiment of formula (V), R^{2d} is selected from chloride and cyano.

According to one embodiment of formula (V), R^{2d} is —CONR^{4d}R^{5d}; and R^{4d} and R^{5d} are the same or different and selected from the group consisting of H and methyl.

According to one embodiment of this aspect of the fourth configuration of the present invention, there is provided a compound selected from the group consisting of:
N-methyl-1-((3-oxoquinuclidin-2-yl)methyl)-1H-1,2,4-triazole-3-carboxamide;
2-((3-chloro-1H-1,2,4-triazol-1-yl)methyl)quinuclidin-3-one;
N,N-dimethyl-1-((3-oxoquinuclidin-2-yl)methyl)-1H-1,2,4-triazole-3-carboxamide;
2-((1H-1,2,4-triazol-1-yl)methyl)quinuclidin-3-one;
1-((3-oxoquinuclidin-2-yl)methyl)-1H-1,2,4-triazole-3-carbonitrile; and
1-((3-oxoquinuclidin-2-yl)methyl)-1H-1,2,4-triazole-3-carboxamide;

or an enantiomer, mixture of enantiomers, pharmaceutically acceptable salt, hydrate, solvate or combination thereof.

According to one embodiment of this aspect of the fourth configuration of the present invention, there is provided a compound selected from the group consisting of:
N-methyl-1-((3-oxoquinuclidin-2-yl)methyl)-1H-1,2,4-triazole-3-carboxamide;
2-((3-chloro-1H-1,2,4-triazol-1-yl)methyl)quinuclidin-3-one;
N,N-dimethyl-1-((3-oxoquinuclidin-2-yl)methyl)-1H-1,2,4-triazole-3-carboxamide;
1-((3-oxoquinuclidin-2-yl)methyl)-1H-1,2,4-triazole-3-carbonitrile; and
1-((3-oxoquinuclidin-2-yl)methyl)-1H-1,2,4-triazole-3-carboxamide;

or an enantiomer, mixture of enantiomers, pharmaceutically acceptable salt, hydrate, solvate or combination thereof.

According to a second aspect of the fourth configuration, the present invention provides a pharmaceutical composition comprising said compound of formula (V) or an enantiomer, mixture of enantiomers, pharmaceutically acceptable salt, hydrate, solvate or combination thereof.

According to a third aspect of the fourth configuration, the present invention provides a compound of formula (V) or an enantiomer, mixture of enantiomers, pharmaceutically acceptable salt, hydrate, solvate or combination thereof according to the first aspect of the fourth configuration, or a pharmaceutical composition according to the second aspect of the fourth configuration, for use in the treatment of a disease associated with a malfunctioning p53 signaling pathway, for example associated with mutant p53.

According to a related, fourth aspect of the fourth configuration, the present invention provides a method of treating a disease associated with a malfunctioning p53 signaling pathway, for example associated with mutant p53, comprising administering a compound of formula (V) or an enantiomer, mixture of enantiomers, pharmaceutically acceptable salt, hydrate, solvate or combination thereof according to the first aspect of the fourth configuration, or a pharmaceutical composition according to the second aspect of the fourth configuration, to a subject in need thereof.

According to a fifth aspect of the fourth configuration, the present invention provides a method of preparing a compound according to the first aspect of the fourth configuration.

Examples of the synthesis of certain compounds of formula (V) are represented in the following Reaction scheme 4:

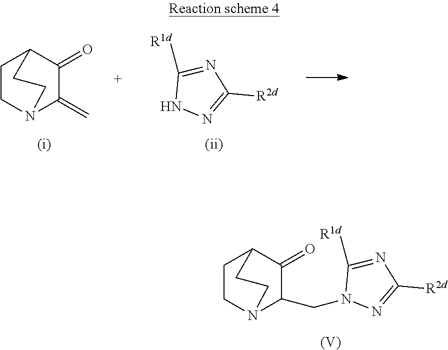

According to the Reaction scheme 4, compound (V) can be made by reacting compound (ii) with 2-methylene-3-quinuclidinone (i) in the presence of an appropriate base in an organic solvent according to the examples below.

Racemic and diastereomeric mixtures as well as single stereoisomers of the disclosed and claimed compounds are within the scope of the present invention.

Further aspects of the invention are defined by the claims and/or are apparent to a person skilled in the art from the disclosure taken as a whole.

As used herein, the term “C₁-C₆ alkyl” means both linear and branched chain saturated hydrocarbon groups with from 1 to 6 carbon atoms. Examples of C₁-C₆ alkyl groups include methyl, ethyl, n-propyl, isopropyl, n-butyl, iso-butyl, sec-butyl, t-butyl, n-pentyl, 1-methyl-butyl, n-hexyl and 2-ethyl-butyl groups. Non-limiting examples of unbranched C₁-C₆ alkyl groups are methyl, ethyl, n-propyl, n-butyl, n-pentyl and n-hexyl groups. Non-limiting examples of branched alkyl groups are iso-propyl, iso-butyl, sec-butyl, t-butyl, 1-methyl-butyl and 2-ethyl-butyl groups.

As used herein, the term “C₁-C₃ alkyl” means both linear and branched chain saturated hydrocarbon groups with from 1 to 3 carbon atoms. Non-limiting examples of C₁-C₃ alkyl groups include methyl, ethyl, n-propyl and isopropyl groups.

As used herein, the term “C₁-C₆ alkoxy” means the group O—C₁-C₆ alkyl, where “C₁-C₆ alkyl” is used as described above. Non-limiting examples of C₁-C₆ alkoxy groups are methoxy, ethoxy, isopropoxy, n-propoxy, n-butoxy, n-hexoxy and 3-methyl-butoxy groups.

As used herein, the term “C₁-C₃ alkoxy” means the group O—C₁-C₃ alkyl, where “C₁-C₃ alkyl” is used as described above. Non-limiting examples of C₁-C₃ alkoxy groups are methoxy, ethoxy, isopropoxy and n-propoxy groups.

As used herein, the term “C₁-C₆ haloalkyl” means both linear and branched chain saturated hydrocarbon groups with from 1 to 6 carbon atoms, having from one to all hydrogens substituted by a halogen of different or same type. Non-limiting examples of C₁-C₆ haloalkyl groups include methyl substituted with from 1 to 3 halogen atoms, ethyl substituted with from 1 to 5 halogen atoms, n-propyl or iso-propyl substituted with from 1 to 7 halogen atoms, n-butyl or iso-butyl substituted with from 1 to 9 halogen atoms, and sec-butyl or t-butyl substituted with from 1 to 9 halogen atoms.

As used herein, the term “C₁-C₃ haloalkyl” means both linear and branched chain saturated hydrocarbon groups with from 1 to 3 carbon atoms, having from one to all hydrogens substituted by a halogen of different or same type. Non-limiting examples of C₁-C₃ haloalkyl groups include methyl substituted with from 1 to 3 halogen atoms, ethyl substituted with from 1 to 5 halogen atoms, and n-propyl or iso-propyl substituted with from 1 to 7 halogen atoms.

As used herein, the term “C₁-C₃ haloalkoxy” means both linear and branched chain saturated alkoxy groups with from 1 to 3 carbon atoms, having from one to all hydrogen atoms substituted by a halogen atom of different or same type. Non-limiting examples of C₁-C₃ haloalkoxy groups include methoxy substituted with from 1 to 3 halogen atoms, ethoxy substituted with from 1 to 5 halogen atoms, and n-propoxy or iso-propoxy substituted with from 1 to 7 halogen atoms.

As used herein, the term “C₁-C₃ fluoroalkyl” means both linear and branched chain saturated hydrocarbon groups with from 1 to 3 carbon atoms, having from one to all hydrogen atoms substituted by a fluorine atom. Non-limiting examples of C₁-C₃ fluoroalkyl groups include methyl substituted with from 1 to 3 fluorine atoms, ethyl substituted with from 1 to 5 fluorine atoms, and n-propyl or iso-propyl substituted with from 1 to 7 fluorine atoms.

As used herein, the term “C₁-C₃ fluoroalkoxy” means both linear and branched chain saturated alkoxy groups with 1 to 3 carbon atoms, having from one to all hydrogen atoms substituted by a fluorine atom. Non-limiting examples of C₁-C₃ fluoroalkoxy groups include methoxy substituted with from 1 to 3 fluorine atoms, ethoxy substituted with from 1 to 5 fluorine atoms, and n-propoxy or iso-propoxy substituted with from 1 to 7 fluorine atoms.

As used herein, the term “C₃-C₆ cycloalkyl” means a cyclic saturated hydrocarbon group with from 3 to 6 carbon atoms. Non-limiting examples of C₃-C₆ cycloalkyl groups include cyclopropyl, cyclobutyl, cyclopentyl and cyclohexyl.

As used herein, the term “C₁-C₃ alkoxy C₁-C₃ alkyl” means both linear and branched chain saturated hydrocarbon groups with 1 to 3 carbon atoms, substituted with an alkoxy group with 1 to 3 carbon atoms. Non-limiting examples of C₁-C₃ alkoxy C₁-C₃ alkyl groups are drawn below.

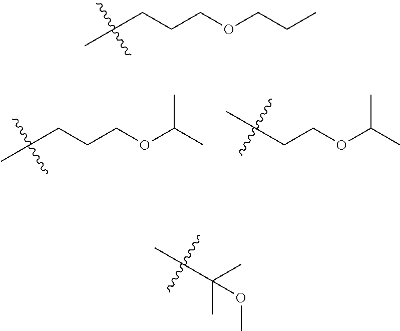

As used herein, the term “C₁-C₃ cyanoalkyl” means both linear and branched chain cyano (CN) derivatives with one to three carbon atoms, including the carbon atom that is part of the cyano group. Non-limiting examples of C₁-C₃ cyanoalkyl groups are drawn below.

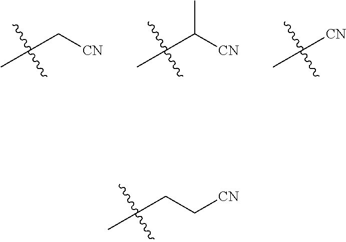

As used herein, the term “N—C₁-C₃ alkylamino” means an C₁-C₃ alkyl substituent attached to the remainder of a molecule via nitrogen. Non-limiting examples of N—C₁-C₃ alkylamino are drawn below.

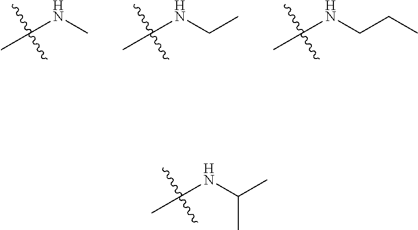

As used herein, the term “N,N-di C₁-C₃ alkylamino” means two C₁-C₃ alkyl substituents attached to the remainder of a molecule via nitrogen. Non-limiting examples of N,N-di C₁-C₃ alkylamino are drawn below.

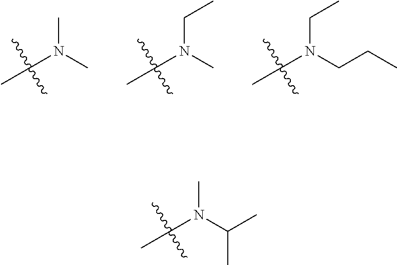

As used herein, the term “amino-C₁-C₃ alkyl” means any amino derivative of a C₁-C₃ alkyl radical. Non-limiting examples of amino-C₁-C₃ alkyl are drawn below.

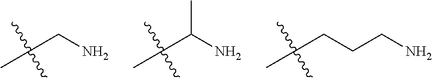

As used herein, the term “halogen” means fluorine, chlorine, bromine or iodine. It is to be understood that when a substituent is halogen (or halo), it is always bound to a carbon atom.

As used herein, the term “aryl” means a monocyclic aromatic carbocyclic group. Non-limiting examples of such groups include phenyl.

As used herein, the term “heteroaryl” means a monocyclic or bicyclic aromatic group of carbon atoms wherein from one to three of the carbon atoms is/are replaced by one or more heteroatoms independently selected from nitrogen, oxygen or sulfur. In a bicyclic heteroaryl, one of the rings may be partially saturated. Non-limiting examples of such groups include indolinyl, dihydrobenzofuranyl and 1,3-benzodioxolyl.

Non-limiting examples of monocyclic heteroaryl groups include furyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl and pyrimidinyl.

Non-limiting examples of bicyclic heteroaryl groups include quinoxalinyl, quinazolinyl, pyridopyrazinyl, benzoxazolyl, benzothiophenyl, benzimidazolyl, naphthyridinyl, quinolinyl, benzofuryl, indolyl, indazolyl, benzothiazolyl, pyridopyrimidinyl and isoquinolinyl.

As used herein, the term “heterocyclyl” means a monocyclic or bicyclic group of carbon atoms wherein from one to three of the carbon atoms is/are replaced by one or more heteroatoms independently selected from nitrogen, oxygen and sulfur. The heterocyclyl group may be further substituted, such as with one or more oxo groups, Non-limiting examples of heterocyclyl groups include 3-oxoquinuclidinyl, tetrahydrofuryl, tetrahydropyranyl, pyrrolidinyl, piperidinyl, piperazinyl, morpholinyl and dioxanyl.

As used herein, and unless specified otherwise, the term “substituted” means that the concerned groups are substituted with at least one functional group, such as hydroxyl, amine, carboxylic acid, halogen, aryl etc. In embodiments, the groups defined above may be optionally further substituted. In embodiments, the groups defined above are not further substituted.

As used herein, and as well-known to persons of skill in the art, a “substituent” means an atom or group that replaces another atom or group in a molecule or can be regarded as replacing an atom in a parent compound.

As such, in the context of the Markush formula (I), Markush formula (II), Markush formula (III), Markush formula (IV) or Markush formula (V) of the compound of the present invention, the substituents R^{1a}, R^{1b}, R^{1c}, R^{1d}, R^{2a}, R^{2b}, R^{2c} and R^{2d} are replaced by the various listed alternative options.

The compounds of the present invention may form salts, which are within the scope of the present invention. Salts of compounds of formula (I), formula (III), formula (III), formula (IV) or formula (V) suitable for use in medicine are for example those wherein a counter ion is pharmaceutically acceptable.

Suitable salts according to the invention include those formed with organic or inorganic acids or bases. In particular, suitable acid addition salts according to the present invention include those formed with mineral acids, strong organic carboxylic acids, or with organic alkyl or aryl sulfonic acids, optionally substituted with halogen.

Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethane-sulfonic, p-toluenesulfonic, formic, benzoic, malonic, naphthalene-2-sulfonic, benzenesulfonic, isethionic, ascorbic, malic, phthalic, aspartic or glutamic acids, as well as from lysine or arginine.

Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucamine, morpholine, thiomorpholine, piperidine, pyrrolidine, a mono, di- or tri lower alkylamine, for example ethyl, tertbutyl, diethyl, diisopropyl, triethyl, tributyl or dimethylpropylamine, or a mono-,di- or trihydroxy lower alkylamine, for example mono-, di- or triethanolamine.

Corresponding internal salts of the compounds of the present invention may furthermore be formed.

In one embodiment of the second aspect of the first, second, third or fourth configuration of the invention, there is provided a pharmaceutical composition comprising a compound according to the first aspect of the first, second, third or fourth configuration of the invention, or an enantiomer, mixture of enantiomers, pharmaceutically acceptable salt, solvate, hydrate or combination thereof, and a pharmaceutically acceptable diluent, carrier and/or excipient.

Such compositions may be suitable for oral or parenteral administration.

In one embodiment, the compounds and compositions of the present invention may be suitable for oral administration. In one embodiment, the compounds and compositions of the present invention may be suitable for parenteral administration, such as intramuscular administration, such as subcutaneous administration, such as intravenous administration.

Compositions for parenteral administration include aqueous and non-aqueous sterile injection solutions which may contain anti-oxidants, buffers or other pH-adjusting components, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as polyethylene glycol, ethanol, 1,3-butanediol, water, Ringer's solution, an isotonic sodium chloride solution, or other suitable dispersing or wetting and suspending agents, including synthetic mono- or diglycerides, and fatty acids, including oleic acid, or Cremophor®.

In aspects of the invention the compound according to the first aspect of the first, second, third or fourth configuration of the invention is intended for use in treatment of a disease associated with a malfunctioning p53 signaling pathway, for example associated with mutant p53.

In one embodiment, such a disease is cancer, as defined in ICD-10, i.e. the tenth revision of the International Classification of Diseases (ICD) maintained by the World Health Organization (WHO), in the categories C00-C97 (malignant neoplasms) and D37-D48 (neoplasms of uncertain or unknown behavior).

Thus in embodiments, there is provided a compound according to the first aspect of the first, second, third or fourth configuration of the invention, or an enantiomer, mixture of enantiomers, pharmaceutically acceptable salt, hydrate, solvate or combination thereof; or a pharmaceutical composition according to the second aspect of the first, second, third or fourth configuration of the invention, for use in the treatment of cancer by administration of said compound or composition to a patient in need thereof.

Typically, said cancer is selected from malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

In one embodiment, said cancer is breast cancer.

In embodiments, the administration of said compound or composition is parenteral.

In embodiments, the administration said compound or composition is in combination with at least one of the following compounds: platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine. ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including, LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PDL2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), an autologous T cells genetically engineered to express a chimeric antigen receptor (CAR) that recognize an extracellular cancer target (including CD19, PSMA, or mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

In embodiments, the administration of said compound or composition is in combination with an external beam irradiation by gamma or neutron radiation or targeted therapy with antibodies labeled with beta or alpha emitting radionuclides, including I-131, Y-90, Lu-177, Bi-213, Ac-225, Th-227, or radiotherapy with Ra-223.

In embodiments, the administration of said compound or composition is in combination with other active pharmaceutical ingredients and further in combination with an external beam irradiation by gamma or neutron radiation or targeted therapy with antibodies labeled with beta or alpha emitting radionuclides, including I-131, Y-90, Lu-177, Bi-213, Ac-225, Th-227, or radiotherapy with Ra-223. Said other active pharmaceutical ingredients may be selected from the group consisting of platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine. ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including, LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PDL2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), an autologous T cells genetically engineered to express a chimeric antigen receptor (CAR) that recognize an extracellular cancer target (including CD19, PSMA, or mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

In embodiments, the administration of said compound or composition in combination with said other active pharmaceutical ingredients is concomitant and/or sequential. In one embodiment, said disease associated with a malfunctioning p53 signaling pathway is selected from autoimmune diseases, for example multiple sclerosis, and cardiac diseases, for example myocardial ischemia.

In one embodiment, said disease is selected from the group consisting of pre-chronic inflammatory diseases, including allergy, asthma, atherosclerosis, coeliac disease, Crohn's disease, gout, inflammatory bowel disease, rheumatoid arthritis and transplant rejection.

In one aspect of the first, second, third or fourth configuration of the invention, there is provided use of a compound according to the first aspect of the first, second, third or fourth configuration of the invention, or an enantiomer, mixture of enantiomers, pharmaceutically acceptable salt, solvate, hydrate or combination thereof in preparing a medicament for treating a disease associated with a malfunctioning p53 signaling pathway, for example associated with mutant p53.

In one aspect of the first, second, third or fourth configuration of the invention, there is provided a method of treating a disease associated with a malfunctioning p53 signaling pathway, for example associated with mutant p53, comprising administering a therapeutically effective amount of a compound according to the present invention, to a patient in need thereof.

In one aspect of the first, second, third or fourth configuration of the invention, there is provided a method of treating a disease associated with a malfunctioning p53 signaling pathway, for example associated with mutant p53, comprising administering a compound according to the first aspect of the first, second, third or fourth configuration of the invention, or an enantiomer, mixture of enantiomers, pharmaceutically acceptable salt, hydrate, solvate or combination thereof; or a pharmaceutical composition according to the second aspect of the first, second, third or fourth configuration of the invention, to a subject in need thereof.

In one embodiment of this aspect, said disease is cancer.

In one embodiment of this aspect, said cancer is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

In one embodiment of this aspect, the administration of said compound of composition is parenteral.

In one embodiment of this aspect, the administration of said compound or composition is in combination with at least one of the following compounds: platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine. ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including, LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PDL2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), an autologous T cells genetically engineered to express a chimeric antigen receptor (CAR) that recognize an extracellular cancer target (including CD19, PSMA, or mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

In one embodiment of this aspect, the administration of said compound or composition is in combination with an external beam irradiation by gamma or neutron radiation or targeted therapy with antibodies labeled with beta or alpha emitting radionuclides, including I-131, Y-90, Lu-177, Bi-213, Ac-225, Th-227, or radiotherapy with Ra-223.

In one embodiment of this aspect, the administration of said compound or composition is in combination with other active pharmaceutical ingredients and further in combination with an external beam irradiation by gamma or neutron radiation or targeted therapy with antibodies labeled with beta or alpha emitting radionuclides, including I-131, Y-90, Lu-177, Bi-213, Ac-225, Th-227, or radiotherapy with Ra-223. Said other active pharmaceutical ingredients may be selected from the group consisting of platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine. ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including, LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PDL2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), an autologous T cells genetically engineered to express a chimeric antigen receptor (CAR) that recognize an extracellular cancer target (including CD19, PSMA, or mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

In one embodiment of this aspect, the administration of said compound or composition in combination with said other active pharmaceutical ingredients is concomitant and/or sequential.

The amount of active ingredient which is required to achieve a therapeutic effect will, of course, vary with the particular compound, the route of administration, the subject under treatment, including the type, species, age, weight, sex, and medical condition of the subject and the renal and hepatic function of the subject, and the particular disorder or disease being treated, as well as its severity. An ordinarily skilled physician, veterinarian or clinician can readily determine and prescribe the effective amount of the drug required to prevent, counter or arrest the progress of the condition.

Oral dosages of the present invention will range from about 0.1 to about 1000 mg per kg of body weight per day (mg/kg/day), preferably from 1 to 500 mg/kg/day, and more preferably from 10 to 250 mg/kg/day, for adult humans. For oral administration, compositions may be provided in the form of tablets or other forms, such as capsules, to provide discrete units containing 0.5, 1.0, 2.5, 5.0, 10.0, 15.0, 25.0, 50.0, 100, 500, 1000, 5000 or 10000 mg of active pharmaceutical ingredient. An oral dosage unit typically contains from about 1 mg to about 5000 mg, preferably from about 1000 mg to about 2500 mg, of active pharmaceutical ingredient.

Parenteral dosages of the present invention, when used for the indicated effects, will range from about 1 to about 1000 mg/kg/day, preferably from 1 to 500 mg/kg/day, most preferably from 10 to 100 mg/kg/day, for adult humans. For intravenous (i.v.) administrations, the most preferred doses will range from about 0.1 to about 10 mg/kg/minute during a constant rate infusion. Compounds and compositions of the present invention may be administered in a single daily dose, or the total daily dosage may be administered in divided doses of two, three or four times daily.

The compounds and compositions of the present invention may be used or administered in combination with at least one of the following compounds (active pharmaceutical ingredients): platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine, ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PD-L2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), autologous T cells genetically engineered to express a chimeric antigen receptor (CAR) that recognize an extracellular cancer target (including CD19, PSMA, mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

Such combined administration may be concomitant and/or sequential.

The compounds and compositions of the present invention may be administered alone or administered in combination with other compounds (active pharmaceutical ingredients), wherein the administration is also in combination with an external beam irradiation by gamma or neutron radiation or targeted therapy with antibodies labeled with beta or alpha emitting radionuclides, including I-131, Y-90, Lu-177, Bi-213, Ac-225 and Th-227, or radiotherapy with Ra-223.

### BRIEF DESCRIPTION OF THE DRAWINGS

FIG. 1 shows the results obtained in Example 34. A: Luciferase luminescence from mice xenografted with MDA-MB-231-Luc breast cancer cells. B: Tumor weight at day 33 after beginning of treatment.

### EXAMPLES

### Example 1: Synthesis of 2,2,2-trichloro-N-ethyl-N-((3-oxoquinuclidin-2-yl)methyl)acetamide

A mixture of 2-methylenequinuclidin-3-one (204 mg, 1.49 mmol), 2 M ethylamine in THF (818 μL, 1.64 mmol) and DIPEA (772 μL, 4.46 mmol) was stirred in DCM (4 mL) for 3 hours. The reaction was cooled to 0° C. and trichloroacetyl chloride (222 μL, 1.99 mmol) was added dropwise. After stirring at 0° C. for 15 minutes LC-MS showed full conversion. The reaction mixture was concentrated and purified by column chromatography on silica gel with MeOH/DCM (1:99) to give the title compound (190 mg, 39%). MS ESI+(m/z): 327, 329, 331 [M+H]+.

### Example 2: Synthesis of 2,2,2-trichloro-N-((3-oxoquinuclidin-2-yl)methyl)acetamide

2-Methylenequinuclidin-3-one (150 mg, 1.09 mmol) was dissolved in DMF (3 mL) and K₂CO₃ (151 mg, 1.09 mmol) was added followed by addition of trichloroacetamide (178 mg, 1.09 mmol). The reaction mixture was stirred at room temperature for 20 hours and at 50° C. for 2 hours to give the desired product, in addition to unreacted starting material. The solids were filtered off and the filtrate was concentrated. The crude material was purified by preparative HPLC (XBridge C18; 50 mM NH₄HCO₃/MeCN; 9:1 to 6:4) to give the title compound (140 mg, 43%). MS ESI+(m/z): 299, 301, 303 [M+H]+.

### Example 3: Synthesis of N-ethyl-2,2,2-trifluoro-N-((3-oxoquinuclidin-2-yl)methyl)acetamide

A mixture of 2-methylenequinuclidin-3-one (159 mg, 1.16 mmol), 2 M ethylamine in THF (580 μL, 1.16 mmol) and K₂CO₃ (160 mg, 1.16 mmol) was stirred in DMF (2 mL) at room temperature for 1 hour and 40 minutes, whereafter the reaction was cooled to 0° C. and trifluoroacetic anhydride (0.24 mL, 1.74 mmol) was added dropwise. The reaction was stirred overnight at room temperature and the solvent was evaporated. The crude product was purified by column chromatography on silica gel with MeOH/DCM (1:99 to 4:96) to give the title compound (67 mg, 21%). MS ESI+(m/z): 279 [M+H]⁺.

### Example 4: Synthesis of 2,2,2-trifluoro-N-((3-oxoquinuclidin-2-yl)methyl)acetamide

A mixture of 2,2,2-trifluoroacetamide (519 mg, 4.59 mmol) and 1.7 M potassium tert-pentoxide in toluene (2.57 mL, 4.37 mmol) was stirred in DMF (15 mL) at room temperature for 5 minutes where after 2-methylenequinuclidin-3-one (600 mg, 4.37 mmol) was added. The reaction was stirred at room temperature for 30 minutes, cooled to 0° C. and quenched by 4M HCl in dioxane (0.98 mL, 3.94 mmol). The solvent was evaporated and the residue was purified by column chromatography on silica gel with MeOH/DCM (1:99 to 3:97). The pure fractions were concentrated to dryness in vacuo to give the title compound (622 mg, 57%) as a white solid. MS ESI+m/z 251 [M+H]⁺.

### Example 5: Synthesis of 2,2-difluoro-N-((3-oxoquinuclidin-2-yl)methyl)acetamide

A mixture of 2,2-difluoroacetamide (72.75 mg, 0.7654 mmol) and 1.7 M potassium tert-pentoxide in toluene (0.43 mL, 0.73 mmol) was stirred in DMF (2.5 mL) at room temperature for 5 minutes. Then, 2-methylenequinuclidin-3-one (100 mg, 0.730 mmol) was added and the reaction was stirred at room temperature for 1 hour and 15 minutes. The reaction was quenched by 4M HCl in dioxane (0.173 mL, 0.692 mmol) and concentrated in vacuo. The crude product was purified by preparative HPLC (XBridge C18; 50 mM NH₄HCO₃/MeCN; 98:2 to 78:22). A second purification was performed by column chromatography on silica gel with MeOH/DCM (0:100 to 15:85). The pure fractions were concentrated and dried under vacuum to afford the title compound (23 mg, 14%) as a white solid. LC-MS ESI+(m/z): 233 [M+H]⁺.

### Comparative Example 1: Synthesis of N-((3-oxoquinuclidin-2-yl)methyl)acetamide

2-Methylenequinuclidin-3-one (100 mg, 0.73 mmol) and acetamide (500 mg, 8.46 mmol) were dissolved in acetonitrile (5 mL) before addition of K₂CO₃ (750 mg, 5.43 mmol). The mixture was heated to 50° C. for 18 hours and then cooled to room temperature. The mixture was diluted with acetonitrile and filtered. The crude mixture was purified by preparative HPLC (XBridge C18; 50 mM NH₄HCO₃/MeCN; 98:2 to 90:10). The pure fractions were concentrated in vacuo to afford the title product (5.6 mg, 4%). LC-MS ESI+(m/z): 197 [M+H]⁺.

### Comparative Example 2: N-((3-oxoquinuclidin-2-yl)methyl)nicotinamide

A suspension of 2-methylenequinuclidin-3-one hydrochloride (50 mg, 0.29 mmol) and pyridine-3-carboxamide (35 mg, 0.29 mmol) in DMF (0.5 mL) was heated at 70° C. After 4 hours, K₂CO₃ (80 mg, 0.58 mmol) was added and heating was continued. After a total of 23 hours the reaction mixture was cooled to room temperature. The reaction mixture was diluted with water (3.5 mL) and purified by preparative HPLC (XBridge C18; 50 mM NH₄HCO₃/MeCN; 98:2 to 60:40). The pure fractions were pooled and concentrated in vacuo to afford the title product as a colorless oil (6 mg, 8%). LC-MS ESI+(m/z): 260 [M+H]⁺.

### Example 6: Synthesis of N-((3-oxoquinuclidin-2-yl)methyl)pyridine-3-sulfonamide

2-Methylenequinuclidin-3-one (137 mg, 1.00 mmol) was added to a solution of pyridine-3-sulfonamide (158 mg, 1.00 mmol) and potassium carbonate (138 mg, 1.00 mmol) in DMF (1 mL) at room temperature. The reaction mixture was stirred for 18 hours and the reaction mixture was poured onto a silica gel column and purified by flash chromatography using DCM/MeOH (100:0 to 85:15). The purest fractions were pooled and concentrated to give a white solid consisting of product and pyridine-3-sulfonamide. The solid was suspended in DCM, filtered and concentrated. The filtrate was purified by preparative HPLC (XBridge C18 19×50 mm; 50 mM NH₄HCO₃/MeCN; 95:5 to 80:20) to give the title compound as a white solid (11 mg. 3.8%). MS ESI+m/z 296 [M+H]⁺.

### Example 7: Synthesis of 4-fluoro-N-((3-oxoquinuclidin-2-yl)methyl)benzenesulfonamide

2-Methylenequinuclidin-3-one (50 mg, 0.36 mmol) was dissolved in DMF (1 mL) followed by addition of 4-fluorophenylsulfonamide (64 mg, 0.36 mmol) and potassium carbonate (50 mg, 0.36 mmol). The reaction mixture was stirred at room temperature for 2 days. After completion 1 eq. HCl in dioxane (4 M, 91 μL, 0.36 mmol) was added and the solvent was evaporated. Purification by preparative HPLC (XBridge C18 19×50 mm; 50 mM NH₄HCO₃/MeCN; 98:2 to 6:4) afforded the title compound (10 mg, 9%) as a white solid. MS ESI+m/z 313 [M+H]⁺.

### Example 8: Synthesis of N-ethyl-N-((3-oxoquinuclidin-2-yl)methyl)methane-sulfonamide

A mixture of 2-methylenequinuclidin-3-one (150 mg, 1.09 mmol), 2 M ethylamine in THF (547 μL, 1.09 mmol) and potassium carbonate (151 mg, 1.09 mmol) in DMF (2 mL) was stirred at room temperature for 1 hour and 40 minutes whereafter the reaction was cooled in an ice bath and MsCl (127 μL, 1.64 mmol) was added. The reaction was stirred for 1 hour whereafter the solvent was evaporated under high vacuum. The crude was purified by column chromatography on silica gel with MeOH/DCM (1:99 to 3:97) to give title compound (19 mg, 6.3%), which solidified upon standing. MS ESI+m/z 261 [M+H]⁺.

### Example 9: Synthesis of N-((3-oxoquinuclidin-2-yl)methyl)methanesulfon-amide

A mixture of methanesulfonamide (71 mg, 0.74 mmol), 2-methylenequinuclidin-3-one (97 mg, 0.71 mmol) and potassium carbonate (89 mg, 0.71 mmol) was stirred in DMF (2.5 mL) at room temperature over night. 4M HCl in dioxane (0.17 mL, 0.68 mmol) was added and the solvent was evaporated. The residue was purified by column chromatography on silica gel with MeOH/DCM (2:98 to 3:97) to give the title compound as a white solid (64 mg, 39%). MS ESI+m/z 233 [M+H]⁺.

### Example 10: Synthesis of N-((3-oxoquinuclidin-2-yl)methyl)benzenesulfon-amide

Potassium carbonate (120.9 mg, 0.875 mmol) and benzenesulfonamide (137.5 mg, 0.875 mmol) were added to a stirring solution of 2-methylenequinuclidin-3-one (120 mg, 0.875 mmol) in DMF (3 mL). The reaction mixture was stirred at room temperature for 5 hours. The solids were filtered off and the filtrate was concentrated. The crude product was purified by preparative HPLC (XBridge C18 19×50 mm, H₂O/MeCN, 98:2 to 80:20) to yield the title compound (120 mg, 47%) LC-MS ESI+m/z 295 [M+H]⁺. Example 11: Synthesis of 2-(N-((3-oxoquinuclidin-2-yl)methyl)methylsulfon-amido)acetamide A mixture of 2-methylenequinuclidin-3-one (104 mg, 0.76 mmol), 2-aminoacetamide hydrochloride (84 mg, 0.76 mmol) and potassium carbonate (157 mg, 1.14 mmol) was stirred in DMF (2 mL) at room temperature for 1 hour and 40 minutes whereafter the reaction was cooled on ice bath and MsCl (88 μL, 1.1 mmol) was added. The reaction was stirred for 30 minutes at room temperature where after the solvent was evaporated. The crude residue product was purified by column chromatography on silica gel with MeOH/DCM (4:96 to 7:93) to give the title compound (5 mg, 2%). MS ESI+m/z 290 [M+H]⁺.

### Example 12: Synthesis of N-(methylsulfonyl)-N-((3-oxoquinuclidin-2-yl)-methyl)qlycine

Benzyl (methylsulfonyl)glycinate (75 mg, 0.31 mmol) and 2-methylenequinuclidin-3-one (85 mg, 0.62 mmol) were dissolved in DMF (1 mL) and potassium carbonate (85.1 mg, 0.61 mmol) was added. The reaction was stirred for 2 hours and the reaction mixture was diluted with DCM (1 mL) and loaded onto a silica gel column preconditioned with DCM. The product was eluted with 2.5% MeOH in DCM and the pure fractions were collected and concentrated to give 17 mg of benzyl N-(methylsulfonyl)-N-((3-oxoquinuclidin-2-yl)methyl)glycinate as an oil. The oil was dissolved in THF (3 mL) and 10% Pd/C (5 mg) was added and the reaction mixture was hydrogenated (1 atm.) overnight. The reaction mixture was diluted with THF (3 mL) and filtered through a sterile filter (0.2 μm). The clear solution was concentrated to give the title compound as a white semisolid (5.0 mg, 39% over two steps). MS ESI+m/z 291 [M+H]⁺.

### Example 13: Synthesis of N-((3-oxoquinuclidin-2-yl)methyl)pyridine-4-sulfon-amide

2-Methylenequinuclidin-3-one (137 mg, 1.00 mmol) was added to a solution of pyridine-4-sulfonamide (158 mg, 1.00 mmol) and potassium carbonate (138 mg, 1.00 mmol) in DMF (1 mL) at room temperature. The reaction mixture was stirred for 5 hours and then stored in the freezer overnight. The crude mixture was purified by column chromatography on silica gel with MeOH/DCM (0:100 to 15:85). The purest fractions were pooled and concentrated. The white solid was suspended in diethylether and filtered to remove unreacted 2-methylenequinuclidin-3-one. The white solid was washed with diethylether (2×1 mL) and dried in vacuo to give the title compound (92 mg, 31%). MS ESI+m/z 296 [M+H]⁺.

### Example 14: Synthesis of N-((3-oxoquinuclidin-2-yl)methyl)pyridine-2-sulfonamide

Pyridine-2-sulfonamide (158.4 mg, 1.00 mmol) was dissolved in DMF (2 mL) and potassium carbonate (138.4 mg, 1.00 mmol) was added. After 5 minutes a solution of 2-methylenequinuclidin-3-one (137.2 mg, 1.00 mmol) in DMF (1 mL) was added. Then the reaction mixture was stirred at room temperature for 5 hours and then placed in the freezer overnight after which LC-MS showed approximately 80% conversion. The solids were removed by filtration and the filtrate was concentrated. The residue was purified by column chromatography on silica gel with MeOH/DCM (1:9). The pure fractions were concentrated to dryness in vacuo to give the title compound (47.3 mg, 16%). LC-MS ESI+m/z 296 [M+H]⁺.

### Example 15: Synthesis of N-ethyl-1,1,1-trifluoro-N-((3-oxoquinuclidin-2-yl)-methyl)methanesulfonamide

A mixture of 2-methylenequinuclidin-3-one (165 mg, 1.20 mmol), 2 M ethylamine in THF (601 μL, 1.20 mmol) and N,N-diisopropylethylamine (0.42 mL, 2.4 mmol) was stirred in DCM (4 mL) at room temperature over night. The mixture was cooled to −78° C. and trifluoromethanesulfonic anhydride (0.22 mL, 1.3 mmol) was added dropwise. The reaction was stirred at −78° C. for 15 minutes whereafter the solvent was evaporated and the crude residue was purified by column chromatography on silica gel with MeOH/DCM (1:99 to 2:98) to give the title compound (16 mg, 4.2%). MS ESI+m/z 315 [M+H]⁺.

### Example 16: Synthesis of 1,1,1-trifluoro-N-((3-oxoquinuclidin-2-yl)methyl)methanesulfonamide

A mixture of trifluoromethanesulfonamide (86 mg, 0.58 mmol) and 1.7 M potassium tert-pentoxide in toluene (0.43 mL, 0.73 mmol) was stirred in DMF (2 mL) at room temperature for 5 minutes whereafter 2-methylenequinuclidin-3-one (100 mg, 0.73 mmol) was added. The reaction was stirred at room temperature for 3 hours and then at 70° C. for 3 days. The reaction was cooled to room temperature and quenched by 4M HCl in dioxane (0.17 mL, 0.69 mmol). The solvent was evaporated and the residue was purified by column chromatography on silica gel with MeOH/DCM (2:98 to 3:97) to give the title compound as a solid (128 mg, 56%). MS ESI+m/z 287 [M+H]⁺.

### Example 17: Synthesis of N,N-bis((3-oxoquinuclidin-2-yl)methyl)methanesulfonamide

2-Methylenequinuclidin-3-one (200 mg, 1.46 mmol) was dissolved in dry THF (2 mL) followed by addition of methanesulfonamide (69.3 mg, 0.73 mmol) and potassium carbonate (202 mg, 1.46 mmol). The mixture was stirred at room temperature overnight and then filtered. The crude product was purified by column chromatography on silica gel with Et₃N/DCM (0.5:99.5) to give the title compound (22 mg, 4%) of an off-white solid after concentration. MS ESI+m/z 370 [M+H]⁺.

### Example 18: Synthesis of N-((3-oxoquinuclidin-2-yl)methyl)propane-2-sulfonamide

A mixture of isopropylsulfonamide (330 mg, 2.68 mmol), potassium carbonate (353 mg, 2.55 mmol) and 2-methylenequinuclidin-3-one (350 mg, 2.55 mmol) was stirred in DMF (8 mL) over night at 60° C. Subsequently, the reaction mixture was cooled on an ice bath and the reaction was quenched by addition of 1M HCl (2.55 mL). The solvent was evaporated and the residue was purified by column chromatography on silica gel with MeOH/DCM (3:97). The fractions containing the product were combined and concentrated to dryness. The product co-eluted with unreacted 2-methylenequinuclidin-3-one which was removed via trituration with diethyl ether. The title compound was obtained as a white solid (171 mg, 26%). MS ESI+m/z 261 [M+H]⁺.

### Example 19: Synthesis of N-((3-oxoquinuclidin-2-yl)methyl)cyclopropane-sulfonamide

2-Methylenequinuclidin-3-one (100 mg, 0.729 mmol) was dissolved in dry DMF (3 mL) followed by addition of cyclopropanesulfonamide (115 mg, 0.949 mmol) and potassium carbonate (101 mg, 0.731 mmol). The mixture was stirred at room temperature for 3 days after which HCl in dioxane (4 M, 365 μL, 1.46 mmol) was added. The mixture was concentrated in vacuo and the product was precipitated with diethyl ether. The solid material was purified by preparative HPLC (XBridge C18 19×50 mm; 50 mM NH₄HCO₃/MeCN; 98:2 to 7:3) to give the crude product as a white solid containing some residual NH₄HCO₃. This was removed by dissolving the product in DCM and filtering off the solid. Concentration yielded the title product (57 mg, 30%). MS ESI+m/z 259 [M+H]⁺.

### Example 20: Synthesis of 1-methyl-N-((3-oxoquinuclidin-2-yl)methyl)cyclo-propane-1-sulfonamide

2-Methylenequinuclidin-3-one (100 mg, 0.729 mmol) was dissolved in dry DMF (2 mL) followed by addition of 1-methylcyclopropane 1-sulfonamide (128 mg, 0.947 mmol) and potassium carbonate (101 mg, 0.731 mmol). The mixture was stirred at room temperature for 4 days after which HCl in dioxane (4 M, 365 μL, 1.46 mmol) was added and the solvent was removed. The product was precipitated and washed with diethyl ether and then purified by preparative HPLC (XBridge C18 19×50 mm; 50 mM NH₄HCO₃/MeCN; 98:2 to 7:3) to give the title compound (67 mg, 34%) as a white solid. MS ESI+m/z 273 [M+H]⁺.

### Example 21: Synthesis of N-cyclopropyl-N-((3-oxoquinuclidin-2-yl)methyl)-methanesulfonamide

A solution of 2-methylenequinuclidin-3-one (150 mg, 1.09 mmol) in DMF (1 mL) was added dropwise to a mixture of cyclopropylamine (0.11 mL, 1.6 mmol) and potassium carbonate (181 mg, 1.31 mmol) in DMF (1 mL). The reaction mixture was stirred for 90 minutes, cooled in an ice/water bath and MsCl (0.17 mL, 2.2 mmol) was added carefully. The reaction mixture was stirred for 1 hour while allowing it to reach ambient temperature. The solvent was evaporated and the crude solid was washed with EtOH. The crude product was purified by column chromatography on silica gel with MeOH/DCM (1:99 to 3:97). The purest fractions were collected and concentrated. The product was crystallized from diethyl ether (2 mL) and washed with diethyl ether (0.5 mL) to give the title compound (7 mg, 2%) as a white solid. MS ESI+m/z 273 [M+H]⁺.

### Example 22: Synthesis of N-((3-oxoquinuclidin-2-yl)methyl)-N-phenyl-methanesulfonamide

2-Methylenequinuclidin-3-one (50 mg, 0.36 mmol) was dissolved in dry DCM (2 mL) followed by addition of N-phenylmethanesulfonamide (62 mg, 0.36 mmol) and potassium carbonate (50 mg, 0.36 mmol). The reaction mixture was stirred for 7 days at room temperature. The solids were filtered off, the solvent evaporated and the residue purified by column chromatography on silica gel with MeOH/EtOAc (0:100 to 2:8) to give the title compound as a colorless oil, which solidified upon standing (47 mg, 42%). MS ESI+m/z 309 [M+H]⁺.

### Example 23: Synthesis of 1-((3-oxoquinuclidin-2-yl)methyl)pyrimidine-2,4(1H,3H)-dione

Pyrimidine-2,4(1H,3H)-dione (164 mg, 1.46 mmol) and potassium carbonate (140 mg, 1.01 mmol) were added to a stirring solution of 2-methylenequinuclidin-3-one (100 mg, 0.729 mmol) in dry DMF (2.5 ml) at room temperature under nitrogen atmosphere. The reaction mixture was stirred at 70° C. for 1 hour. Ice cold water (0.5 mL) was added to the reaction mixture and the white precipitate was filtered off and washed with heptane. The crude solid was suspended in DCM (50 mL) and stirred for 15 minutes at 35° C. to remove insoluble uracil. The suspension was filtered and the filtrate was concentrated in vacuo to give the title compound as a white solid (9.9 mg, 5%). MS ESI+(m/z): 250 [M+H]⁺.

### Example 24: Synthesis of 5-methyl-1-((3-oxoquinuclidin-2-yl)methyl)pyrimidine-2,4(1H,3H)-dione

To a suspension of 5-methyl-1H-pyrimidine-2,4-dione (9.19 g, 72.9 mmol) in anhydrous DMF (200 mL) potassium carbonate (20.15 g, 145.8 mmol) was added while stirring. After 10 minutes, a solution of 2-methylenequinuclidin-3-one (10.0 g, 72.9 mmol) in DMF (25 mL) was added dropwise using an addition funnel, followed by rinsing the addition funnel with 25 mL DMF. Stirring was continued for 1.5 hours at room temperature to get full conversion according to LCMS. Upon acidification with 3M HCl (aq.) the product dissolved and the inorganic salts were filtered off. Subsequently, the pH was adjusted to 7 and the mixture was concentrated under reduced pressure. The residue was suspended in 120 mL of water and stirred for 10 minutes. The solid material filtered off and washed with diethyl ether to give the title compound (11.43 g, 60%) as a white solid. MS ESI+(m/z): 264 [M+H]⁺.

### Example 25: Synthesis of tert-butyl 5-methyl-2,6-dioxo-3-((3-oxoquinuclidin-2-yl)methyl)-3,6-dihydropyrimidine-1(2H)-carboxylate

A mixture of 2-methylenequinuclidin-3-one (118 mg, 0.86 mmol), tert-butyl 5-methyl-2,4-dioxo-1H-pyrimidine-3-carboxylate (195 mg, 0.86 mmol) and potassium carbonate (119 mg, 0.86 mmol) in DMF (3 mL) was stirred at room temperature over night. 1 M HCl (0.86 mL, 0.86 mmol) was added and the solvent was evaporated. To the residue were added DCM and MgSO₄. The solid was filtered off and the solvent evaporated. The crude residue was purified by column chromatography on silica gel eluting first with DCM/EtOAc (8:2) to remove unreacted BOC protected thymine reactant and then with DCM/MeOH (99:1 to 97:3) to give the title compound (245 mg, 78%). MS ESI+m/z 364 [M+H]⁺.

### Example 26: Synthesis of 5-methyl-1,3-bis((3-oxoquinuclidin-2-yl)methyl)-pyrimidine-2,4(1H,3H)-dione

A mixture of 2-methylenequinuclidin-3-one (175 mg, 1.28 mmol), 5-methyl-1H-pyrimidine-2,4-dione (73 mg, 0.58 mmol) and potassium carbonate (160 mg, 1.16 mmol) in DMF (4 mL) was stirred at 60° C. over night. To the mixture was added 1 M HCl (1.16 mL, 1.16 mmol) followed by evaporation of the solvent. The crude product was purified by column chromatography on silica gel with DCM/MeOH/NH₃ (28% aq.) (91:7:2) to give the title compound (180 mg, 78%). The title compound was isolated as a mixture of diastereomers. MS ESI+(m/z): 401 [M+H]⁺.

### Comparative Example 3: Synthesis of 5-fluoro-1-((3-oxoquinuclidin-2-yl)methyl)pyrimidine-2,4(H,3H)-dione

5-fluorouracil (94.8 mg, 0.730 mmol) was dissolved in dry THF (5 mL) followed by addition of 2-methylene-3-quinuclidinone (100 mg, 0.730 mmol) and K₂CO₃ (201.2 mg, 1.46 mmol). Dry DMF (1 mL) was added to dissolve 5-fluorouracil. After 2 days, the mixture was filtered and the filtrate washed with dichloromethane. The filter material was purified by preparative HPLC (XBridge C18; 50 mM NH₄HCO₃/MeCN; 98:2 to 80:20) to give the product as a white solid (67 mg, 34%). MS ESI+(m/z) 268 [M+H]⁺.

### Example 27: Synthesis of N-methyl-1-((3-oxoquinuclidin-2-yl)methyl)-1H-1,2,4-triazole-3-carboxamide

Potassium carbonate (101 mg, 0.731 mmol) was added to a solution of 2-methylenequinuclidin-3-on (100 mg, 0.729 mmol) in DMF (2.5 mL) followed by addition of N-methyl-1H-1,2,4-triazole-3-carboxamide (91.9 mg, 0.729 mmol). The mixture was stirred at room temperature over night. The solids were filtered off and the reaction mixture was concentrated under vacuum. The residue was purified by preparative HPLC (XBridge C18 19×50 mm; 50 mM NH₄HCO₃/MeCN; 95:5 to 75:25). The fractions containing the product were concentrated and dissolved in DCM. The solution was washed with water and then concentrated. The concentrate was dissolved in diethyl ether and filtered through a 0.45 μm polypropylene syringe filter. The mixture was concentrated and then dissolved in EtOH, filtered and concentrated to give the title compound (1.5 mg, 0.8%). MS ESI+(m/z): 264 [M+H]⁺.

### Example 28: Synthesis of 2-((3-chloro-1H-1,2,4-triazol-1-yl)methyl)quinuclidin-3-one

A mixture of 3-chloro-1H-1,2,4-triazole (83 mg, 0.80 mmol), 2-methylenequinuclidin-3-one (105 mg, 0.77 mmol) and potassium carbonate (97 mg, 0.70 mmol) was stirred in DMF (2.5 mL) at room temperature over night. 1 M HCl (0.73 mL, 0.73 mmol) was added and the solvent was evaporated. The residue was purified by column chromatography on silica gel with MeOH/DCM (2:98 to 3:97) to give the title compound as a white solid (67 mg, 36%). MS ESI+(m/z): 241 [M+H]⁺.

### Example 29: Synthesis of N,N-dimethyl-1-((3-oxoquinuclidin-2-yl)methyl)-1H-1,2,4-triazole-3-carboxamide

Potassium carbonate (78.9 mg, 0.571 mmol) was added to a solution of 2-methylenequinuclidin-3-one (78.3 mg, 0.571 mmol) in DMF (2.5 mL) followed by addition of N,N-dimethyl-1H-1,2,4-triazole-3-carboxamide (80.0 mg, 0.571 mmol). The mixture was stirred at room temperature over night. LCMS analysis of the reaction mixture revealed the formation of two isomeric products in a ratio of 1:3. The solids were filtered off and the reaction mixture was concentrated under vacuum. The residue was purified by column chromatography on silica gel with MeOH/DCM (1:9 to 2:8) and the title compound was isolated as the major and second eluting isomer (20 mg, 13%). MS ESI+(m/z): 278 [M+H]⁺.

### Example 30: Synthesis of 2-((1H-1,2,4-triazol-1-yl)methyl)quinuclidin-3-one

A mixture of 2-methylenequinuclidin-3-one (100 mg, 0.729 mmol), 1H-1,2,4-triazole (151 mg, 2.19 mmol) and potassium carbonate (101 mg, 0.731 mmol) was stirred in DMF (2.5 mL) at 70° C. over night. The solid material was filtered off and the solvent was evaporated. The crude residue was purified by preparative HPLC (XBridge C18 19×50 mm; 50 mM NH₄HCO₃/MeCN; 99.5:0.5 to 95:5). The pure fractions were pooled and lyophilized to give the title compound (45 mg, 30%). MS ESI+(m/z): 207 [M+H]⁺.

### Example 31: Synthesis of 1-((3-oxoquinuclidin-2-yl)methyl)-1H-1,2,4-triazole-3-carbonitrile

Potassium carbonate (100.8 mg, 0.729 mmol) was added to a stirring solution of 1H-1,2,4-triazole-3-carbonitrile (68.8 mg, 0.731 mmol) in DMF (2.5 mL) at room temperature. After 15 minutes, 2-methylenequinuclidin-3-one (100 mg, 0.729 mmol) was added. The reaction mixture was stirred at room temperature for 6 hours after which approximately 60% conversion of starting material was obtained according to LCMS. The solids were filtered off and solvent was concentrated in vacuo. The crude residue was purified by preparative HPLC (XBridge C18 19×50 mm; 50 mM NH₄HCO₃/MeCN; 99:1 to 85:15). The fractions containing the product were concentrated and then stirred in diethyl ether in order to precipitate 2-methylenequinuclidin-3-one. The solids were filtered off and the filtrate concentrated. This procedure was repeated to give the pure title compound (55 mg, 33%). MS ESI+(m/z): 232 [M+H]⁺.

### Example 32: Synthesis of 1-((3-oxoquinuclidin-2-yl)methyl)-1H-1,2,4-triazole-3-carboxamide

Potassium tert-pentoxide (1.7 M in toluene, 430 μL, 0.73 mmol) was added to a stirring solution of 1H-1,2,4-triazole-3-carboxamide (89.88 mg, 0.802 mmol) in DMF (2.5 mL) at room temperature. The mixture was heated to 60° C. for 15 minutes followed by addition of 2-methylenequinuclidin-3-one (100 mg, 0.729 mmol). The reaction mixture was stirred at 60° C. for 1 hour and then cooled in an ice/water bath and the reaction was quenched with 4M HCl in dioxane (182 μL, 0.73 mmol). The solvent was removed in vacuo and the residue was purified by preparative HPLC (XBridge C18 19×50 mm; 50 mM NH₄HCO₃/MeCN; 99:1 to 9:1) to give the title compound (105 mg, 58%). MS ESI+(m/z): 250 [M+H]⁺.

### Comparative Example 4: Synthesis of 2-((1H-pyrazol-1-yl)methyl)quinuclidin-3-one

1H-Pyrazole (49.6 mg, 0.730 mmol) and K₂CO₃ (100.8 mg, 0.730 mmol) were added to a stirring solution of 2-methylenequinuclidin-3-one (100 mg, 0.730 mmol) in DMF (2.5 mL) at room temperature. The reaction mixture was stirred at 70° C. for 1 hour and the cooled to room temperature. The solids were filtered off and the solvent was removed in vacuo. The crude residue was purified by preparative HPLC (XBridge C18, 50 mM NH₄HCO₃/MeCN; 98:2 to 60:40) to give title compound (35 mg, 23%). LC-MS ESI+(m/z): 206 [M+H]⁺.

### Comparative Example 5: Synthesis of 2-((1H-1,2,3-triazol-5-yl)methyl)quinuclidin-3-one

1H-Triazole (85 μL, 1.5 mmol) and K₂CO₃ (100.8 mg, 0.730 mmol) were added to a stirring solution of 2-methylenequinuclidin-3-one (100 mg, 0.730 mmol) in DMF (2.5 mL) at room temperature. The reaction mixture was stirred at room temperature for 3 hours. The solids were filtered off and solvent was removed in vacuo. The crude residue was purified by preparative HPLC (XBridge C18, 50 mM NH₄HCO₃/MeCN; 99.5:0.5 to 87:13) to give the title compound (16 mg, 11%). LC-MS ESI+(m/z): 207 [M+H]⁺. Two isomers were formed in the reaction, but only one was isolated in a pure form. The structure of the isolated isomer was confirmed by NMR to be that of Comparative example 5.

The exemplified compounds of Examples 1-32 and Comparative examples 1-5 above were all synthesized, isolated and tested as a racemic mixture, unless stated otherwise.

### Example 33: In Vitro Efficacy Study Usinq the SaOS-2 His273 Protocol

SaOS-2 His273 mtp53 is a human osteosarcoma cell line which has been genetically engineered at OnkPat CCK to express His273 mutated p53.

3000 cells/well (50 μl) were seeded into masked (black or white with clear bottom) 96-well cell culture plates using Iscove's Modified Dulbecco's Medium supplemented with 10% heat inactivated (56° C. for 60 min) fetal calf serum. The plates were then incubated for 4 hours, allowing the cells to attach. The test compounds were dissolved in DMSO or water to a concentration of 0.01 M and then further diluted to desired concentrations using the cell medium. 50 μl of freshly diluted test compound from fresh stock was added to the wells. The plates were then incubated for 72 hours.

For viability detection, CellTiter-Glo® Luminescent Cell Viability Assay (CTG) was used. CTG is a homogeneous “add-mix-measure” format method of determining the number of viable cells in culture based on quantitation of the ATP present, an indicator of metabolically active cells. The luminescence was measured using the PerkinElmer Victorx4 instrument.

The average of the signal values for the untreated cells was calculated for each plate. The % of growth suppression was calculated as:

100−((Signal sample/Signal untreated cells)×100).

The results of the SaOs-2 His273 analyses were expressed as IC50 values, i.e. concentration that suppresses growth of at least 50% of the cells. The IC50 values of various compounds according to the invention and of some comparative compounds are shown in Table 1, Table 2, Table 3 and Table 4.

TABLE 1
Cell based activity in SaOS-2 His273

| | | SaOS-2 IC50 |
|---|---|---|
| Example | Structure | His273 (μM) |
| 1 | 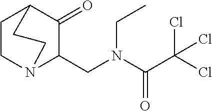 [SMILES: CCN(CC1C(=O)C2CCN1CC2)C(=O)C(Cl)(Cl)Cl] | 3.2 |
| 2 | 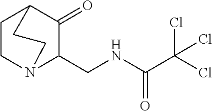 [SMILES: O=C1C2CCN(CC2)C1CNC(=O)C(Cl)(Cl)Cl] | 6.9 |
| 3 | 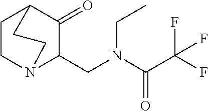 [SMILES: CCN(CC1C(=O)C2CCN1CC2)C(=O)C(F)(F)F] | 4.2 |
| 4 | 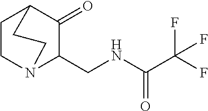 [SMILES: O=C1C2CCN(CC2)C1CNC(=O)C(F)(F)F] | 5.6 |
| 5 | 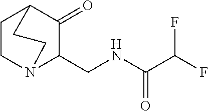 [SMILES: O=C(NCC1C(=O)C2CCN1CC2)C(F)F] | 13 |
| Comparative | | SaOS-2 IC50 |
| example | Structure | His273 (μM) |
| 1 | 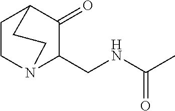 | >40 |
| 2 | 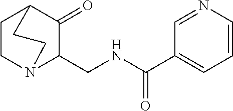 [SMILES: O=C(NCC1C(=O)C2CCN1CC2)c1cccnc1] | >40 |

TABLE 2
Cell based activity in SaOS-2 His273

| | | SaOS-2 IC50 |
|---|---|---|
| Example | Structure | His273 (μM) |
| 6 | 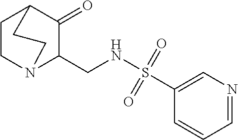 [SMILES: O=C1C2CCN(CC2)C1CNS(=O)(=O)c1cccnc1] | 7.2 |
| 7 | 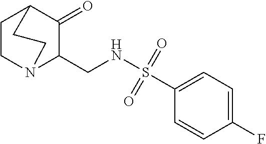 [SMILES: O=C1C2CCN(CC2)C1CNS(=O)(=O)c1ccc(F)cc1] | 7.2 |
| 8 | 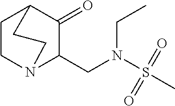 [SMILES: CCN(CC1C(=O)C2CCN1CC2)S(C)(=O)=O] | 6.0 |
| 9 | 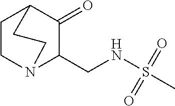 | 8.0 |
| 10 | 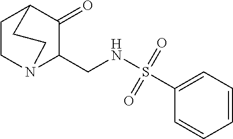 [SMILES: O=C1C2CCN(CC2)C1CNS(=O)(=O)c1ccccc1] | 7.1 |
| 11 | 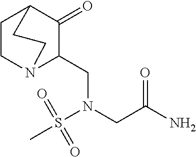 [SMILES: CS(=O)(=O)N(CC(N)=O)CC1C(=O)C2CCN1CC2] | 4.1 |
| 12 | 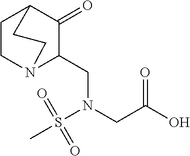 [SMILES: CS(=O)(=O)N(CC(=O)O)CC1C(=O)C2CCN1CC2] | 6.0 |
| 13 | 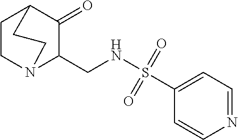 [SMILES: O=C1C2CCN(CC2)C1CNS(=O)(=O)c1ccncc1] | 6.3 |
| 14 | 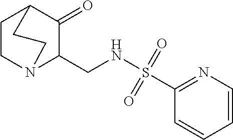 [SMILES: O=C1C2CCN(CC2)C1CNS(=O)(=O)c1ccccn1] | 6.0 |
| 15 | 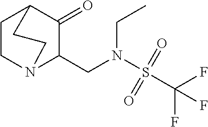 [SMILES: CCN(CC1C(=O)C2CCN1CC2)S(=O)(=O)C(F)(F)F] | 3.5 |
| 16 | 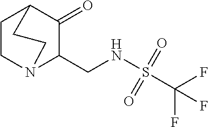 [SMILES: O=C1C2CCN(CC2)C1CNS(=O)(=O)C(F)(F)F] | 2.9 |
| 17 | 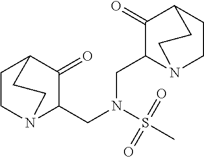 [SMILES: CS(=O)(=O)N(CC1C(=O)C2CCN1CC2)CC1C(=O)C2CCN1CC2] | 2.9 |
| 18 | 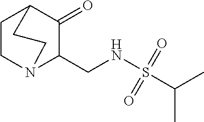 [SMILES: CC(C)S(=O)(=O)NCC1C(=O)C2CCN1CC2] | 11 |
| 19 | 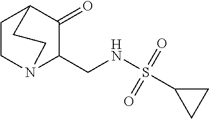 [SMILES: O=C1C2CCN(CC2)C1CNS(=O)(=O)C1CC1] | 12 |
| 20 | 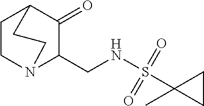 [SMILES: CC1(S(=O)(=O)NCC2C(=O)C3CCN2CC3)CC1] | 7.4 |
| 21 | 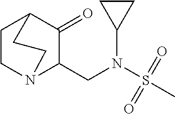 [SMILES: CS(=O)(=O)N(CC1C(=O)C2CCN1CC2)C1CC1] | 1.8 |
| 22 | 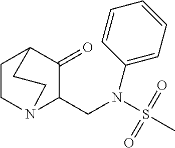 [SMILES: CS(=O)(=O)N(CC1C(=O)C2CCN1CC2)c1ccccc1] | 1.6 |

TABLE 3
Cell based activity in SaOS-2 His273

| | | SaOS-2 IC50 |
|---|---|---|
| Example | Structure | His273 (μM) |
| 23 | 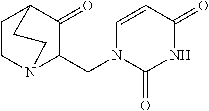 [SMILES: O=C1C2CCN(CC2)C1Cn1ccc(=O)[nH]c1=O] | 3.2 |
| 24 | 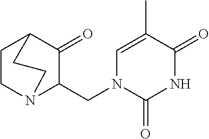 [SMILES: Cc1cn(CC2C(=O)C3CCN2CC3)c(=O)[nH]c1=O] | 6.5 |
| 25 | 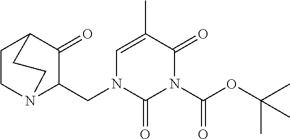 [SMILES: Cc1cn(CC2C(=O)C3CCN2CC3)c(=O)n(C(=O)OC(C)(C)C)c1=O] | 5.3 |
| 26 | 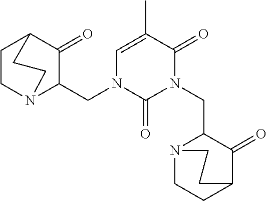 [SMILES: Cc1cn(CC2C(=O)C3CCN2CC3)c(=O)n(CC2C(=O)C3CCN2CC3)c1=O] | 4.0 |

TABLE 4
Cell based activity in SaOS-2 His273

| | | SaOS-2 IC50 |
|---|---|---|
| Example | Structure | His273 (μM) |
| 27 | 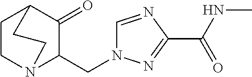 [SMILES: CNC(=O)c1ncn(CC2C(=O)C3CCN2CC3)n1] | 5.0 |
| 28 | 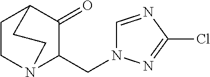 [SMILES: O=C1C2CCN(CC2)C1Cn1cnc(Cl)n1] | 3.1 |
| 29 | 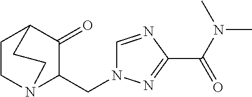 [SMILES: CN(C)C(=O)c1ncn(CC2C(=O)C3CCN2CC3)n1] | 8.8 |
| 30 | 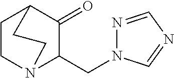 [SMILES: O=C1C2CCN(CC2)C1Cn1cncn1] | 26 |
| 31 | 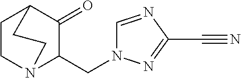 [SMILES: N#Cc1ncn(CC2C(=O)C3CCN2CC3)n1] | 2.4 |
| 32 | 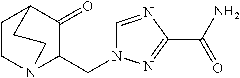 [SMILES: NC(=O)c1ncn(CC2C(=O)C3CCN2CC3)n1] | 10 |
| Comparative | | SaOS-2 IC50 |
| example | Structure | His273 (μM) |
| 4 | 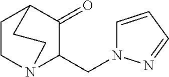 [SMILES: O=C1C2CCN(CC2)C1Cn1cccn1] | >40 |
| 5 | 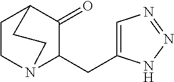 [SMILES: O=C1C2CCN(CC2)C1Cc1cnn[nH]1] | >40 |

### Example 34: In Vivo Efficacy Study

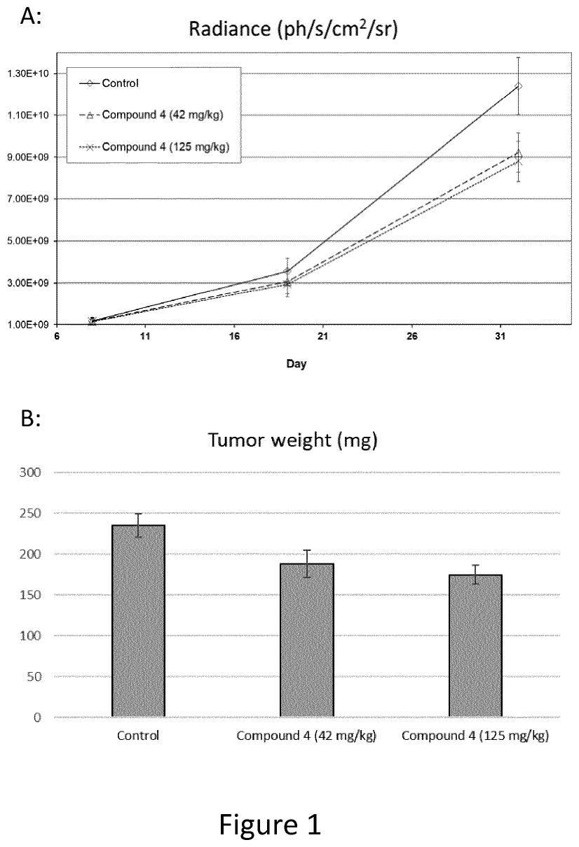

The compound of Example 4 was administered intraperitoneally twice daily at 42 or 125 mg/kg to nude mice (Hsd:AthymicNude-Foxnlnu) ortotopically xenografted with the human breast cancer cell line MDA-MB-231-Luc, carrying mutant p53 (R280K). To make MDA-MB-231-Luc, the cell line MDA-MB-231 was stably transfected with a synthetic gene for firefly luciferase, Luc2 (pGL4, Promega). Thereby, the tumor load may be externally monitored by luminescence measurement (radiance values; photon/second/cm²/steradian). 5×10⁶ MBA-MB-231-luc cells were injected, and eight days later the animals were stratified into treatment groups and control, 8 animals in each group, based on luminescence measurement. Treatment was started on day 11 and done in 3 cycles of 5 days each, with 2 days recovery. Tumor progression was evaluated by non-invasive in vivo imaging during the second cycle (day 18) and at the end of treatment (day 32). On day 33, the animals were sacrificed and the tumors excised and weighed. The result is shown in FIG. 1. From FIG. 1A, it is clear that treatment with the compound of Example 4 reduced tumor cell migration compared to control animals, as measured by radiance of luminescence. FIG. 1B demonstrates that treatment with the same compound reduced tumor weight compared to control animals.

### Example 35: In Vivo Efficacy Study

An example of Table 2, an example of Table 3 or an example of Table 4 is administered intraperitoneally twice daily at approximately 42 or 125 mg/kg to nude mice (Hsd:AthymicNude-Foxnlnu) ortotopically xenografted with the human breast cancer cell line MDA-MB-231-Luc, carrying mutant p53 (R280K). To make MDA-MB-231-Luc, the cell line MDA-MB-231 is stably transfected with a synthetic gene for firefly luciferase, Luc2 (pGL4, Promega). Thereby, the tumor load may be externally monitored by luminescence measurement (radiance values; photon/second/cm²/steradian). 5×10⁶ MBA-MB-231-luc cells are injected, and eight days later the animals are stratified into treatment groups and control, 8 animals in each group, based on luminescence measurement. Treatment is started on day 11 and done in 3 cycles of 5 days each, with 2 days recovery. Tumor progression is evaluated by non-invasive in vivo imaging during the second cycle (day 18) and at the end of treatment (day 32). On day 33, the animals are sacrificed and the tumors excised and weighed.

### Example 36: Stability Study

Chemical stability at low pH was investigated for selected compounds. Compounds that are highly unstable may not be suitable as drug candidates, for example due to potential difficulties in achieving and maintaining a stable and therapeutically effective formulation. For instance, compounds intended for oral administration must be chemically stable at the low pH observed in the stomach in order for this to be an acceptable route.

A pH 4 buffer was prepared by mixing 4.1 mL of 0.2 M acetic acid (aq.) with 0.9 mL of a 0.2 M sodium acetate (aq.) and then diluting to 10 mL with distilled water. The pH was determined by pH meter to 4.04.

Benzylpiperidine-4-ol was used as internal standard. A 1 mg/mL stock solution was prepared by dissolving 3.5 mg benzylpiperidine-4-ol in 3.5 mL of water. 500 μL of the stock solution were added to pH 4 buffer (5 mL) and the mixture was shaken.

A 1 mg/mL solution in acetonitrile was prepared for each test compound. Two drops of water were added to ca. 1 mL of acetonitrile in order to completely dissolve Example 2.

To 400 μL of pH 4 buffer with internal standard were added 100 μL of 1 mg/mL test compound solution. The solutions were stored at 25° C. and analyzed by LCMS directly and at different time points up to 48 hours. The [M+H]⁺ ion for parent compound (test compound) and the internal standard (m/z 192) were extracted and integrated. The area for the parent compound was normalized to the area of the internal standard for each time point.

The half-life was calculated based on 1^{st} order kinetics (t %=ln2/k). The rate constant (k) was obtained by plotting In[normalized peak area] against time.

Results for half-life and percent parent compound remaining at 48 h for selected examples and a comparative example are shown in Table 5.

TABLE 5
Stability study in pH 4 buffer at 25° C.

| | % Parent remaining at 48 h | Half-life (h) |
|---|---|---|
| Example | | |
| 23 | 59 | 67 |
| 24 | >95 | >630 |
| 26 | 73 | 70 |
| Comparative | | |
| example | | |
| 3 | 18 | 15 |

1. A compound of formula (I) 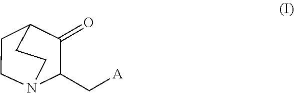
wherein
A represents 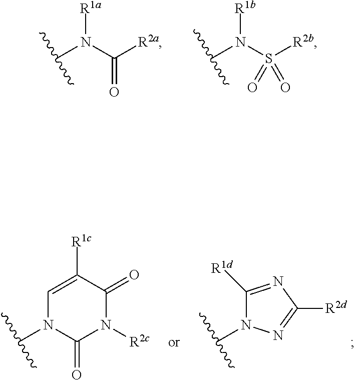
R^{1a} is selected from the group consisting of H, C₁-C₆ alkyl, C₁-C₆ haloalkyl, C₃-C₆ cycloalkyl and C₃-C₆ cyclohaloalkyl, said alkyl, haloalkyl, cycloalkyl and cyclohaloalkyl being optionally substituted with one or more C₁-C₆ alkoxy;
R^{2a} is C₁-C₆ haloalkyl;
R^{1b} is selected from the group consisting of H, C₁-C₆ alkyl, C₁-C₆ haloalkyl, C₃-C₆ cycloalkyl, C₃-C₆ cyclohaloalkyl, phenyl, halogenated phenyl, benzyl, halogenated benzyl and —CH₂—R^{3b}, said alkyl, haloalkyl, cycloalkyl, cyclohaloalkyl, phenyl and halogenated phenyl being optionally substituted with one or more C₁-C₃ alkyl, C₁-C₃ haloalkyl, C₁-C₆ alkoxy or C₁-C₆ haloalkoxy;
R^{2b} is selected from the group consisting of C₁-C₆ alkyl, C₁-C₆ haloalkyl, C₃-C₆ cycloalkyl, C₃-C₆ cyclohaloalkyl, phenyl, halogenated phenyl, benzyl, halogenated benzyl, heteroaryl and halogenated heteroaryl, said alkyl, haloalkyl, cycloalkyl, cyclohaloalkyl, phenyl, halogenated phenyl, benzyl, halogenated benzyl, heteroaryl and halogenated heteroaryl being optionally substituted with one or more C₁-C₃ alkyl, C₁-C₃ haloalkyl, C₁-C₆ alkoxy or C₁-C₆ haloalkoxy;
R^{3b} is selected from the group consisting of heterocyclyl, COOR^{4b} and CONR^{5b}R^{6b};
R^{4b} is selected from the group consisting of H, C₁-C₆ alkyl and C₁-C₆ haloalkyl;
R^{5b} and R^{6b} are the same or different and are selected from the group consisting of H, C₁-C₆ alkyl and C₁-C₆ haloalkyl;
R^{1c} is selected from the group consisting of H, C₁-C₆ alkyl, C₁-C₆ haloalkyl, C₃-C₆ cycloalkyl and C₃-C₆ cyclohaloalkyl, said alkyl, haloalkyl, cycloalkyl and cyclohaloalkyl being optionally substituted with one or more C₁-C₆ alkoxy;
R^{2c} is selected from the group consisting of H, —CH₂—R^{3c} and —COOR^{4c};
R^{3c} is heterocyclyl;
R^{4c} is selected from the group consisting of C₁-C₆ alkyl and C₁-C₆ haloalkyl;
R^{1d} is selected from the group consisting of H, C₁-C₆ alkyl, C₁-C₆ haloalkyl, C₃-C₆ cycloalkyl and C₃-C₆ cyclohaloalkyl, said alkyl, haloalkyl, cycloalkyl and cyclohaloalkyl being optionally substituted with one or more C₁-C₆ alkoxy or halogen; and
R^{2d} is selected from the group consisting of H, halogen, cyano, —COOR^{3d} and —CONR^{4d}R^{5d};
R^{3d} is selected from the group consisting of H, C₁-C₆ alkyl and C₁-C₆ haloalkyl; and
R^{4d} and R^{5d} are the same or different and selected from the group consisting of H, C₁-C₆ alkyl and C₁-C₆ haloalkyl;
or an enantiomer, mixture of enantiomers, pharmaceutically acceptable salt, hydrate, solvate or combination thereof.

2. A compound according to claim 1, wherein said compound is of formula (II) 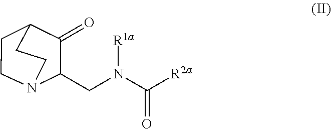
wherein
R^{1a} is selected from the group consisting of H, C₁-C₆ alkyl, C₁-C₆ haloalkyl, C₃-C₆ cycloalkyl and C₃-C₆ cyclohaloalkyl, said alkyl, haloalkyl, cycloalkyl and cyclohaloalkyl being optionally substituted with one or more C₁-C₆ alkoxy; and
R^{2a} is C₁-C₆ haloalkyl;
or an enantiomer, mixture of enantiomers, pharmaceutically acceptable salt, hydrate, solvate or combination thereof.

3. A compound according to claim 2, wherein
R^{1a} is selected from the group consisting of H, C₁-C₆ alkyl and C₁-C₆ haloalkyl, said alkyl and haloalkyl being optionally substituted with one or more C₁-C₆ alkoxy; and
R^{2a} is C₁-C₆ haloalkyl;
or an enantiomer, mixture of enantiomers, pharmaceutically acceptable salt, hydrate, solvate or combination thereof.

4. A compound according to claim 2 or 3, wherein
R^{1a} is selected from the group consisting of H and C₁-C₆ alkyl, said alkyl being optionally substituted with one or more C₁-C₆ alkoxy; and
R^{2a} is C₁-C₆ haloalkyl;
or an enantiomer, mixture of enantiomers, pharmaceutically acceptable salt, hydrate, solvate or combination thereof.

5. A compound according to any one of claims 2-4, wherein
R^{1a} is selected from the group consisting of H and ethyl; and
R^{2a} is selected from the group consisting of trihalomethyl and dihalomethyl;
or an enantiomer, mixture of enantiomers, pharmaceutically acceptable salt, hydrate, solvate or combination thereof.

6. A compound according to claim 2, wherein R^{1a} is H, or an enantiomer, mixture of enantiomers, pharmaceutically acceptable salt, hydrate, solvate or combination thereof.

7. A compound according to claim 2, wherein R^{1a} is ethyl, or an enantiomer, mixture of enantiomers, pharmaceutically acceptable salt, hydrate, solvate or combination thereof.

8. A compound according to claim 2, wherein R^{2a} is selected from the group consisting of CF₃ and CCl₃, or an enantiomer, mixture of enantiomers, pharmaceutically acceptable salt, hydrate, solvate or combination thereof.

9. A compound according to claim 2, selected from the group consisting of:
2,2,2-trichloro-N-ethyl-N-((3-oxoquinuclidin-2-yl)methyl)acetamide;
2,2,2-trichloro-N-((3-oxoquinuclidin-2-yl)methyl)acetamide;
N-ethyl-2,2,2-trifluoro-N-((3-oxoquinuclidin-2-yl)methyl)acetamide;
2,2,2-trifluoro-N-((3-oxoquinuclidin-2-yl)methyl)acetamide; and
2,2-difluoro-N-((3-oxoquinuclidin-2-yl)methyl)acetamide,
or an enantiomer, mixture of enantiomers, pharmaceutically acceptable salt, hydrate, solvate or combination thereof.

10. A pharmaceutical composition comprising
a compound according to any one of claims 2-9, or an enantiomer, mixture of enantiomers, pharmaceutically acceptable salt, hydrate, solvate or combination thereof; and
a pharmaceutically acceptable diluent, carrier and/or excipient.

11. A compound according to any one of claims 2-9, or an enantiomer, mixture of enantiomers, pharmaceutically acceptable salt, hydrate, solvate or combination thereof; or a pharmaceutical composition according to claim 10, for use in the treatment of cancer by administration of said compound or composition to a patient in need thereof.

12. Compound or composition for use according to claim 11, wherein said cancer is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

13. Compound or composition for use according to claim 11 or 12, wherein the administration is parenteral.

14. Compound or composition for use according to any one of claims 11-13, wherein the administration is in combination with at least one of the following compounds: platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine, ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including, LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PDL2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), an autologous T cells genetically engineered to express a chimeric antigen receptor (CAR) that recognize an extracellular cancer target (including CD19, PSMA, or mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

15. Compound or composition for use according to any one of claims 11-13, wherein the administration is alone or in combination with other active pharmaceutical ingredients and wherein the administration is optionally also in combination with an external beam irradiation by gamma or neutron radiation or targeted therapy with antibodies labeled with beta or alpha emitting radionuclides, including I-131, Y-90, Lu-177, Bi-213, Ac-225, Th-227, or radiotherapy with Ra-223.

16. Compound or composition for use according to claim 14 or 15, wherein the administration is concomitant and/or sequential.

17. A method of treating a disease associated with a malfunctioning p53 signaling pathway, for example associated with mutant p53, comprising administering a compound according to any one of claims 2-9, or an enantiomer, mixture of enantiomers, pharmaceutically acceptable salt, hydrate, solvate or combination thereof, or a pharmaceutical composition according to claim 10, to a subject in need thereof.

18. A method according to claim 17, wherein said disease is cancer.

19. A method according to claim 18, wherein said cancer is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

20. A method according to any one of claims 17-19, wherein the administration is parenteral.

21. A method according to any one of claims 17-20, wherein the administration is in combination with at least one of the following compounds: platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine, ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including, LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PDL2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), an autologous T cells genetically engineered to express a chimeric antigen receptor (CAR) that recognize an extracellular cancer target (including CD19, PSMA, or mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

22. A method according to any one of claims 17-20, wherein the administration is alone or in combination with other active pharmaceutical ingredients and wherein the administration is optionally also in combination with an external beam irradiation by gamma or neutron radiation or targeted therapy with antibodies labeled with beta or alpha emitting radionuclides, including I-131, Y-90, Lu-177, Bi-213, Ac-225, Th-227, or radiotherapy with Ra-223.

23. A method according to claim 21 or 22, wherein the administration is concomitant and/or sequential

24. A compound according to claim 1, wherein said compound is of formula (III) 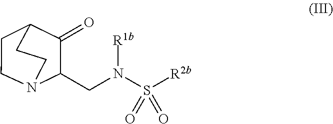
wherein
R^{1b} is selected from the group consisting of H, C₁-C₆ alkyl, C₁-C₆ haloalkyl, C₃-C₆ cycloalkyl, C₃-C₆ cyclohaloalkyl, phenyl, halogenated phenyl, benzyl, halogenated benzyl and —CH₂—R^{3b}, said alkyl, haloalkyl, cycloalkyl, cyclohaloalkyl, phenyl and halogenated phenyl being optionally substituted with one or more C₁-C₃ alkyl, C₁-C₃ haloalkyl, C₁-C₆ alkoxy or C₁-C₆ haloalkoxy;
R^{2b} is selected from the group consisting of C₁-C₆ alkyl, C₁-C₆ haloalkyl, C₃-C₆ cycloalkyl, C₃-C₆ cyclohaloalkyl, phenyl, halogenated phenyl, benzyl, halogenated benzyl, heteroaryl and halogenated heteroaryl, said alkyl, haloalkyl, cycloalkyl, cyclohaloalkyl, phenyl, halogenated phenyl, benzyl, halogenated benzyl, heteroaryl and halogenated heteroaryl being optionally substituted with one or more C₁-C₃ alkyl, C₁-C₃ haloalkyl, C₁-C₆ alkoxy or C₁-C₆ haloalkoxy;
R^{3b} is selected from the group consisting of heterocyclyl, COOR^{4b} and CONR^{5b}R^{6b};
R^{4b} is selected from the group consisting of H, C₁-C₆ alkyl and C₁-C₆ haloalkyl; and
R^{5b} and R^{6b} are the same or different and are selected from the group consisting of H, C₁-C₆ alkyl and C₁-C₆ haloalkyl;
or an enantiomer, mixture of enantiomers, pharmaceutically acceptable salt, hydrate, solvate or combination thereof.

25. A compound according to claim 24, wherein R^{1b} is selected from the group consisting of H, C₁-C₆ alkyl, C₃-C₆ cycloalkyl, phenyl and —CH₂—R^{3b}, said alkyl, cycloalkyl and phenyl being optionally substituted with one or more C₁-C₆ alkoxy or C₁-C₆ haloalkoxy;
or an enantiomer, mixture of enantiomers, pharmaceutically acceptable salt, hydrate, solvate or combination thereof.

26. A compound according to claim 24 or 25, wherein R^{1b} is selected from the group consisting of H, ethyl, —CH₂-(3-oxoquinuclidin-2-yl), CH₂CONH₂, CH₂CO₂H, cyclopropyl and phenyl;
or an enantiomer, mixture of enantiomers, pharmaceutically acceptable salt, hydrate, solvate or combination thereof.

27. A compound according to any one of claims 24-26, wherein R^{2b} is selected from the group consisting of C₁-C₆ alkyl, C₁-C₆ haloalkyl, C₃-C₆ cycloalkyl, phenyl, halogenated phenyl and heteroaryl, said alkyl, cycloalkyl, phenyl, and heteroaryl being optionally substituted with one or more C₁-C₃ alkyl, C₁-C₃ haloalkyl, C₁-C₆ alkoxy or C₁-C₆ haloalkoxy;
or an enantiomer, mixture of enantiomers, pharmaceutically acceptable salt, hydrate, solvate or combination thereof.

28. A compound according to any one of claims 24-27, wherein R^{2b} is selected from the group consisting of C₁-C₆ alkyl, C₁-C₆ haloalkyl, C₃-C₆ cycloalkyl, phenyl, halogenated phenyl and heteroaryl;
or an enantiomer, mixture of enantiomers, pharmaceutically acceptable salt, hydrate, solvate or combination thereof.

29. A compound according to claim 24, wherein R^{2b} is selected from the group consisting of methyl, trifluoromethyl, isopropyl, cyclopropyl, 1-methylcyclopropyl, phenyl, 4-fluorphenyl, 2-pyridinyl, 3-pyridinyl and 4-pyridinyl;
or an enantiomer, mixture of enantiomers, pharmaceutically acceptable salt, hydrate, solvate or combination thereof.

30. A compound according to any one of claims 24-29, wherein R^{3b} is selected from the group consisting of 3-oxoquinuclidin-2-yl, COOR^{4b} and CONR⁵R^{6b};
or an enantiomer, mixture of enantiomers, pharmaceutically acceptable salt, hydrate, solvate or combination thereof.

31. A compound according to claim 24 selected from the group consisting of:
N-((3-oxoquinuclidin-2-yl)methyl)pyridine-3-sulfonamide;
4-fluoro-N-((3-oxoquinuclidin-2-yl)methyl)benzenesulfonamide;
N-ethyl-N-((3-oxoquinuclidin-2-yl)methyl)methanesulfonamide;
N-((3-oxoquinuclidin-2-yl)methyl)methanesulfonamide;
N-((3-oxoquinuclidin-2-yl)methyl)benzenesulfonamide;
2-(N-((3-oxoquinuclidin-2-yl)methyl)methylsulfonamido)acetamide;
N-(methylsulfonyl)-N-((3-oxoquinuclidin-2-yl)methyl)glycine;
N-((3-oxoquinuclidin-2-yl)methyl)pyridine-4-sulfonamide;
N-((3-oxoquinuclidin-2-yl)methyl)pyridine-2-sulfonamide;
N-ethyl-1,1,1-trifluoro-N-((3-oxoquinuclidin-2-yl)-methyl)methanesulfonamide;
1,1,1-trifluoro-N-((3-oxoquinuclidin-2-yl)methyl)methanesulfonamide;
N,N-bis((3-oxoquinuclidin-2-yl)methyl)methanesulfonamide;
N-((3-oxoquinuclidin-2-yl)methyl)propane-2-sulfonamide;
N-((3-oxoquinuclidin-2-yl)methyl)cyclopropanesulfonamide;
1-methyl-N-((3-oxoquinuclidin-2-yl)methyl)cyclopropane-1-sulfonamide;
N-cyclopropyl-N-((3-oxoquinuclidin-2-yl)methyl)methanesulfonamide; and
N-((3-oxoquinuclidin-2-yl)methyl)-N-phenylmethanesulfonamide;
or an enantiomer, mixture of enantiomers, pharmaceutically acceptable salt, hydrate, solvate or combination thereof.

32. A pharmaceutical composition comprising
a compound according to any one of claims 24-31, or an enantiomer, mixture of enantiomers, pharmaceutically acceptable salt, hydrate, solvate or combination thereof; and
a pharmaceutically acceptable diluent, carrier and/or excipient.

33. A compound according to any one of claims 24-31, or an enantiomer, mixture of enantiomers, pharmaceutically acceptable salt, hydrate, solvate or combination thereof; or a pharmaceutical composition according to claim 32, for use in the treatment of cancer by administration of said compound or composition to a patient in need thereof.

34. Compound or composition for use according to claim 33, wherein said cancer is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

35. Compound or composition for use according to claim 33 or 34, wherein the administration is parenteral.

36. Compound or composition for use according to any one of claims 33-35, wherein the administration is in combination with at least one of the following compounds: platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine, ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including, LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PDL2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), an autologous T cells genetically engineered to express a chimeric antigen receptor (CAR) that recognize an extracellular cancer target (including CD19, PSMA, or mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

37. Compound or composition for use according to any one of claims 33-35, wherein the administration is alone or in combination with other active pharmaceutical ingredients and wherein the administration is optionally also in combination with an external beam irradiation by gamma or neutron radiation or targeted therapy with antibodies labeled with beta or alpha emitting radionuclides, including I-131, Y-90, Lu-177, Bi-213, Ac-225, Th-227, or radiotherapy with Ra-223.

38. Compound or composition for use according to claim 36 or 37, wherein the administration is concomitant and/or sequential.

39. A method of treating a disease associated with a malfunctioning p53 signaling pathway, for example associated with mutant p53, comprising administering a compound according to any one of claims 24-31, or an enantiomer, mixture of enantiomers, pharmaceutically acceptable salt, hydrate, solvate or combination thereof, or a pharmaceutical composition according to claim 32, to a subject in need thereof.

40. A method according to claim 39, wherein said disease is cancer.

41. A method according to claim 40, wherein said cancer is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

42. A method according to any one of claims 39-41, wherein the administration is parenteral.

43. A method according to any one of claims 39-42, wherein the administration is in combination with at least one of the following compounds: platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine, ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including, LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PDL2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), an autologous T cells genetically engineered to express a chimeric antigen receptor (CAR) that recognize an extracellular cancer target (including CD19, PSMA, or mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

44. A method according to any one of claims 39-42, wherein the administration is alone or in combination with other active pharmaceutical ingredients and wherein the administration is optionally also in combination with an external beam irradiation by gamma or neutron radiation or targeted therapy with antibodies labeled with beta or alpha emitting radionuclides, including I-131, Y-90, Lu-177, Bi-213, Ac-225, Th-227, or radiotherapy with Ra-223.

45. A method according to claim 43 or 44, wherein the administration is concomitant and/or sequential.

46. A compound according to claim 1, wherein said compound is of formula (IV) 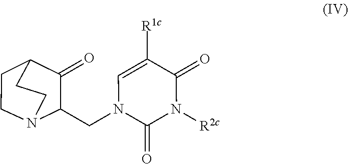
wherein
R^{1c} is selected from the group consisting of H, C₁-C₆ alkyl, C₁-C₆ haloalkyl, C₃-C₆ cycloalkyl and C₃-C₆ cyclohaloalkyl, said alkyl, haloalkyl, cycloalkyl and cyclohaloalkyl being optionally substituted with one or more C₁-C₆ alkoxy;
R^{2c} is selected from the group consisting of H, —CH₂—R^{3c} and —COOR^{4c};
R^{3c} is heterocyclyl; and
R^{4c} is selected from the group consisting of C₁-C₆ alkyl and C₁-C₆ haloalkyl;
or an enantiomer, mixture of enantiomers, pharmaceutically acceptable salt, hydrate, solvate or combination thereof.

47. A compound according to claim 46, wherein
R^{1c} is selected from the group consisting of H, C₁-C₆ alkyl and C₁-C₆ haloalkyl, said alkyl and haloalkyl being optionally substituted with one or more C₁-C₆ alkoxy;
R^{2c} is selected from the group consisting of H, —CH₂—R^{3c} and —COOR^{4c};
R^{3c} is heterocyclyl; and
R^{4c} is selected from the group consisting of C₁-C₆ alkyl and C₁-C₆ haloalkyl;
or an enantiomer, mixture of enantiomers, pharmaceutically acceptable salt, hydrate, solvate or combination thereof.

48. A compound according to claim 46 or 47, wherein
R^{1c} is selected from the group consisting of H and C₁-C₆ alkyl;
R^{2c} is selected from the group consisting of H, —CH₂—R^{3c} and —COOR^{4c};
R^{3c} is heterocyclyl; and
R^{4c} is selected from the group consisting of C₁-C₆ alkyl and C₁-C₆ haloalkyl;
or an enantiomer, mixture of enantiomers, pharmaceutically acceptable salt, hydrate, solvate or combination thereof.

49. A compound according to any one of claims 46-48, wherein
R^{1c} is selected from the group consisting of H and methyl;
R^{2c} is selected from the group consisting of H, —CH₂—R^{3c} and —COOR^{4c};
R^{3c} is heterocyclyl; and
R^{4c} is tert-butyl;
or an enantiomer, mixture of enantiomers, pharmaceutically acceptable salt, hydrate, solvate or combination thereof.

50. A compound according to any one of claims 46-49, wherein
R^{1c} is selected from the group consisting of H and methyl;
R^{2c} is selected from the group consisting of H, —CH₂—R^{3c} and —COOR^{4c};
R^{3c} is 3-oxoquinuclidin-2-yl; and
R^{4c} is tert-butyl;
or an enantiomer, mixture of enantiomers, pharmaceutically acceptable salt, hydrate, solvate or combination thereof.

51. A compound according to claim 46 wherein R^{1c} is H;
or an enantiomer, mixture of enantiomers, pharmaceutically acceptable salt, hydrate, solvate or combination thereof.

52. A compound according to claim 46 wherein R^{1c} is methyl;
or an enantiomer, mixture of enantiomers, pharmaceutically acceptable salt, hydrate, solvate or combination thereof.

53. A compound according to claim 46, selected from the group consisting of:
1-((3-oxoquinuclidin-2-yl)methyl)pyrimidine-2,4(1H,3H)-dione;
5-methyl-1-((3-oxoquinuclidin-2-yl)methyl)pyrimidine-2,4(1H,3H)-dione;
tert-butyl 5-methyl-2,6-dioxo-3-((3-oxoquinuclidin-2-yl)methyl)-3,6-dihydropyrimidine-1(2H)-carboxylate; and
5-methyl-1,3-bis((3-oxoquinuclidin-2-yl)methyl)pyrimidine-2,4(1H,3H)-dione;
or an enantiomer, mixture of enantiomers, pharmaceutically acceptable salt, hydrate, solvate or combination thereof.

54. A pharmaceutical composition comprising
a compound according to any one of claims 46-53, or an enantiomer, mixture of enantiomers, pharmaceutically acceptable salt, hydrate, solvate or combination thereof; and
a pharmaceutically acceptable diluent, carrier and/or excipient.

55. A compound according to any one of claims 46-53, or an enantiomer, mixture of enantiomers, pharmaceutically acceptable salt, hydrate, solvate or combination thereof; or a pharmaceutical composition according to claim 54, for use in the treatment of cancer by administration of said compound or composition to a patient in need thereof.

56. Compound or composition for use according to claim 55, wherein said cancer is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

57. Compound or composition for use according to claim 55 or 56, wherein the administration is parenteral.

58. Compound or composition for use according to any one of claims 55-57, wherein the administration is in combination with at least one of the following compounds: platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine, ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including, LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PDL2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), an autologous T cells genetically engineered to express a chimeric antigen receptor (CAR) that recognize an extracellular cancer target (including CD19, PSMA, or mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

59. Compound or composition for use according to any one of claims 55-57, wherein the administration is alone or in combination with other active pharmaceutical ingredients and wherein the administration is optionally also in combination with an external beam irradiation by gamma or neutron radiation or targeted therapy with antibodies labeled with beta or alphaemitting radionuclides, including I-131, Y-90, Lu-177, Bi-213, Ac-225, Th-227, or radiotherapy with Ra-223.

60. Compound or composition for use according to claim 58 or 59, wherein the administration is concomitant and/or sequential.

61. A method of treating a disease associated with a malfunctioning p53 signaling pathway, for example associated with mutant p53, comprising administering a compound according to any one of claims 46-53, or an enantiomer, mixture of enantiomers, pharmaceutically acceptable salt, hydrate, solvate or combination thereof, or a pharmaceutical composition according to claim 54, to a subject in need thereof.

62. A method according to claim 61, wherein said disease is cancer.

63. A method according to claim 62 wherein said cancer is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

64. A method according to any one of claims 61-63, wherein the administration is parenteral.

65. A method according to any one of claims 61-64, wherein the administration is in combination with at least one of the following compounds: platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine, ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including, LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PDL2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), an autologous T cells genetically engineered to express a chimeric antigen receptor (CAR) that recognize an extracellular cancer target (including CD19, PSMA, or mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

66. A method according to any one of claims 61-64, wherein the administration is alone or in combination with other active pharmaceutical ingredients and wherein the administration is optionally also in combination with an external beam irradiation by gamma or neutron radiation or targeted therapy with antibodies labeled with beta or alpha emitting radionuclides, including I-131, Y-90, Lu-177, Bi-213, Ac-225, Th-227, or radiotherapy with Ra-223.

67. A method according to claim 64 or 65, wherein the administration is concomitant and/or sequential.

68. A compound according to claim 1, wherein said compound is of formula (V) 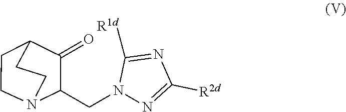
wherein
R^{1d} is selected from the group consisting of H, C₁-C₆ alkyl, C₁-C₆ haloalkyl, C₃-C₆ cycloalkyl and C₃-C₆ cyclohaloalkyl, said alkyl, haloalkyl, cycloalkyl and cyclohaloalkyl being optionally substituted with one or more C₁-C₆ alkoxy or halogen; and
R^{2d} is selected from the group consisting of H, halogen, cyano, —COOR^{3d} and —CONR^{4d}R^{5d};
R^{3d} is selected from the group consisting of H, C₁-C₆ alkyl and C₁-C₆ haloalkyl; and
R^{4d} and R^{5d} are the same or different and selected from the group consisting of H, C₁-C₆ alkyl and C₁-C₆ haloalkyl;
or an enantiomer, mixture of enantiomers, pharmaceutically acceptable salt, hydrate, solvate or combination thereof.

69. A compound according to claim 68, wherein
R^{1d} is selected from the group consisting of H, C₁-C₆ alkyl and C₁-C₆ haloalkyl, said alkyl and haloalkyl being optionally substituted with one or more C₁-C₆ alkoxy;
or an enantiomer, mixture of enantiomers, pharmaceutically acceptable salt, hydrate, solvate or combination thereof.

70. A compound according to claim 68 or 69, wherein
R^{1d} is H;
R^{2d} is selected from the group consisting of H, halogen, cyano and —CONR^{4d}R^{5d}; and
R^{4d} and R^{5d} are the same or different and selected from the group consisting of H and C₁-C₆ alkyl;
or an enantiomer, mixture of enantiomers, pharmaceutically acceptable salt, hydrate, solvate or combination thereof.

71. A compound according to any one of claims 68-70, wherein
R^{1d} is H;
R^{2d} is selected from the group consisting of H, chloride, cyano and —CONR^{4d}R^{5d}; and
R^{4d} and R^{5d} are the same or different and selected from the group consisting of H and methyl;
or an enantiomer, mixture of enantiomers, pharmaceutically acceptable salt, hydrate, solvate or combination thereof.

72. A compound according to claim 68 wherein R^{1d} is H;
or an enantiomer, mixture of enantiomers, pharmaceutically acceptable salt, hydrate, solvate or combination thereof.

73. A compound according to claim 68 wherein R^{2d} is selected from H, chloride and cyano;
or an enantiomer, mixture of enantiomers, pharmaceutically acceptable salt, hydrate, solvate or combination thereof.

74. A compound according to claim 68 wherein
R^{2d} is —CONR^{4d}R^{5d}; and
R^{4d} and R^{5d} are the same or different and selected from the group consisting of H and methyl;
or an enantiomer, mixture of enantiomers, pharmaceutically acceptable salt, hydrate, solvate or combination thereof.

75. A compound according to claim 68, selected from the group consisting of:
N-methyl-1-((3-oxoquinuclidin-2-yl)methyl)-1H-1,2,4-triazole-3-carboxamide;
2-((3-chloro-1H-1,2,4-triazol-1-yl)methyl)quinuclidin-3-one;
N,N-dimethyl-1-((3-oxoquinuclidin-2-yl)methyl)-1H-1,2,4-triazole-3-carboxamide;
2-((1H-1,2,4-triazol-1-yl)methyl)quinuclidin-3-one;
1-((3-oxoquinuclidin-2-yl)methyl)-1H-1,2,4-triazole-3-carbonitrile; and
1-((3-oxoquinuclidin-2-yl)methyl)-1H-1,2,4-triazole-3-carboxamide;
or an enantiomer, mixture of enantiomers, pharmaceutically acceptable salt, hydrate, solvate or combination thereof.

76. A pharmaceutical composition comprising
a compound according to any one of claims 68-75, or an enantiomer, mixture of enantiomers, pharmaceutically acceptable salt, hydrate, solvate or combination thereof; and
a pharmaceutically acceptable diluent, carrier and/or excipient.

77. A compound according to any one of claims 68-75, or an enantiomer, mixture of enantiomers, pharmaceutically acceptable salt, hydrate, solvate or combination thereof; or a pharmaceutical composition according to claim 76, for use in the treatment of cancer by administration of said compound or composition to a patient in need thereof.

78. Compound or composition for use according to claim 77, wherein said cancer is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

79. Compound or composition for use according to claim 77 or 78, wherein the administration is parenteral.

80. Compound or composition for use according to any one of claims 77-79, wherein the administration is in combination with at least one of the following compounds: platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine, ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including, LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PDL2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), an autologous T cells genetically engineered to express a chimeric antigen receptor (CAR) that recognize an extracellular cancer target (including CD19, PSMA, or mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

81. Compound or composition for use according to any one of claims 77-79, wherein the administration is alone or in combination with other active pharmaceutical ingredients and wherein the administration is optionally also in combination with an external beam irradiation by gamma or neutron radiation or target3ed therapy with antibodies labeled with beta or alphaemitting radionuclides, including I-131, Y-90, Lu-177, Bi-213, Ac-225, Th-227, or radiotherapy with Ra-223.

82. Compound or composition for use according to claim 80 or 81, wherein the administration is concomitant and/or sequential.

83. A method of treating a disease associated with a malfunctioning p53 signaling pathway, for example associated with mutant p53, comprising administering a compound according to any one of claims 68-75, or an enantiomer, mixture of enantiomers, pharmaceutically acceptable salt, hydrate, solvate or combination thereof, or a pharmaceutical composition according to claim 76, to a subject in need thereof.

84. A method according to claim 83, wherein said disease is cancer.

85. A method according to claim 84 wherein said cancer is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

86. A method according to any one of claims 83-85, wherein the administration is parenteral.

87. A method according to any one of claims 83-86, wherein the administration is in combination with at least one of the following compounds: platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine, ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including, LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PDL2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), an autologous T cells genetically engineered to express a chimeric antigen receptor (CAR) that recognize an extracellular cancer target (including CD19, PSMA, or mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

88. A method according to any one of claims 83-86, wherein the administration is alone or in combination with other active pharmaceutical ingredients and wherein the administration is optionally also in combination with an external beam irradiation by gamma or neutron radiation or targeted therapy with antibodies labeled with beta or alpha emitting radionuclides, including I-131, Y-90, Lu-177, Bi-213, Ac-225, Th-227, or radiotherapy with Ra-223.

89. A method according to claim 87 or 88, wherein the administration is concomitant and/or sequential.

90. A pharmaceutical composition comprising
a compound according to claim 1, or an enantiomer, mixture of enantiomers, pharmaceutically acceptable salt, hydrate, solvate or combination thereof; and
a pharmaceutically acceptable diluent, carrier and/or excipient.

91. A compound according to claim 1, or an enantiomer, mixture of enantiomers, pharmaceutically acceptable salt, hydrate, solvate or combination thereof; or a pharmaceutical composition according to claim 90, for use in the treatment of cancer by administration of said compound or composition to a patient in need thereof.

92. Compound or composition for use according to claim 91, wherein said cancer is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

93. Compound or composition for use according to claim 91 or 92, wherein the administration is parenteral.

94. Compound or composition for use according to any one of claims 91-93, wherein the administration is in combination with at least one of the following compounds: platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine, ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including, LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PDL2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), an autologous T cells genetically engineered to express a chimeric antigen receptor (CAR) that recognize an extracellular cancer target (including CD19, PSMA, or mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

95. Compound or composition for use according to any one of claims 91-93, wherein the administration is alone or in combination with other active pharmaceutical ingredients and wherein the administration is optionally also in combination with an external beam irradiation by gamma or neutron radiation or target3ed therapy with antibodies labeled with beta or alphaemitting radionuclides, including I-131, Y-90, Lu-177, Bi-213, Ac-225, Th-227, or radiotherapy with Ra-223.

96. Compound or composition for use according to claim 94 or 95, wherein the administration is concomitant and/or sequential.

97. A method of treating a disease associated with a malfunctioning p53 signaling pathway, for example associated with mutant p53, comprising administering a compound according to claim 1, or an enantiomer, mixture of enantiomers, pharmaceutically acceptable salt, hydrate, solvate or combination thereof, or a pharmaceutical composition according to claim 90, to a subject in need thereof.

98. A method according to claim 97 wherein said disease is cancer.

99. A method according to claim 98 wherein said cancer is selected from the group consisting of malignant neoplasms, stated or presumed to be primary, of the following sites: malignant neoplasms of lip, oral cavity and pharynx including head and neck cancer; malignant neoplasms of digestive organs including esophagus, colon, liver or pancreas cancer; malignant neoplasms of respiratory and intrathoracic organs including lung cancer; malignant neoplasms of bone and articular cartilage including osteosarcoma; melanoma and other malignant neoplasms of skin; malignant neoplasms of mesothelial and soft tissue including sarcoma; malignant neoplasm of breast; malignant neoplasms of female genital organs including ovarian cancer; malignant neoplasms of male genital organs including prostate cancer; malignant neoplasms of urinary tract including bladder cancer; malignant neoplasms of eye, brain and other parts of central nervous system including glioblastoma; malignant neoplasms of thyroid and other endocrine glands including thyroid cancer; malignant neoplasms of ill-defined, secondary and unspecified sites; malignant neoplasms of lymphoid, hematopoietic and related tissue including multiple myeloma, lymphoid leukemia or myeloid leukemia; neoplasms of uncertain or unknown behavior including myelodysplastic syndrome.

100. A method according to any one of claims 97-99, wherein the administration is parenteral.

101. A method according to any one of claims 97-100, wherein the administration is in combination with at least one of the following compounds: platinum based antineoplastic agents (including cisplatin, carboplatin, dicycloplatin, nedaplatin, oxaliplatin, picoplatin, satraplatin), nucleoside analogs and antimetabolites (including cytarabine, fludarabine, gemcitabine, 5FU), DNA intercalators (including danorubicin, doxorubicin, epirubicin and idarubicin, camptothecin), alkylating neoplastic agents (including cyclophosphamide, melphalan, bendamustine, carmustine, lomustine, ifosfamide), topoisomerase inhibitors (including etoposide, topotecan), PARP inhibitors (including olaparib, niraparib, rucaparib), a substance interfering with microtubule dynamics (including combrestatin, eribulin, docetaxel, taxane, vinoblastine, vincristine), a substance blocking the interaction between p53 and MDM2 or MDM4 (including nutlins, idasanutlin, HDM-201, DS3032b, AMG-232, ALRN-6924), a kinase inhibitor (including BRAF inhibitors vemurafenib, dabrafenib), a PI3K and/or mTOR inhibitor (including, LY294002, dactolisib, rapamycin and rapamycin analogs temsirolimus, everolimus, ridaforolimus), an MRP1 inhibitor (including indomethacin, meloxicam, sulindac sulfide, GSK1904529A, MK571, verapamil), hypomethylation agents (including azacitidine, decitabine), histone deacetylase inhibitor (including cirtuins, hydroxamates including vorinostat, belinostat, dacinostat, panobinostat, valproic acid, benzamides including entinostat, mocetinostat), proteasome inhibitors (including bortezomib, ritonavir, carfilzomib), an antivascular or antiangiogenic agent (including 2aG4, bevacizumab), tyrosine kinase inhibitor (including lapatinib), EGFR inhibitors (including gefitinib), CDK inhibitors, PLK inhibitors, MEK inhibitors (including pimasertib), immune checkpoint inhibitors (including antibodies against PD-1 (including nivolumab, pembrolizumab), PD-L1 (avelumab, atezolizumab), PDL2, CTLA-4 (including ipilimumab, tremelimumab), GITR, IL-40, CD-40, LAG3/CD-223 (including BMS-986016, REGN3767), OX-40 (including pogalizumab, PF-04518600)), antibodies binding protein tyrosine kinase receptors, NFE2L2 inhibitors (including ML385, brusatol, trigonelline, luteolin, ascorbic acid, ATRA), an autologous T cells genetically engineered to express a chimeric antigen receptor (CAR) that recognize an extracellular cancer target (including CD19, PSMA, or mesothelin), glucocorticoid receptor agonist (including dexamethasone), buthionine sulfoximine, folic acid, metformin, sorafenib, sulfasalazine, bleomycin, erlotinib, tunicamycin, wortmannin, pidilizumab, durvalumab, GSK3174998, tavolixizumab, deazaneplanocin A or piperlongumine.

102. A method according to any one of claims 97-109, wherein the administration is alone or in combination with other active pharmaceutical ingredients and wherein the administration is optionally also in combination with an external beam irradiation by gamma or neutron radiation or targeted therapy with antibodies labeled with beta or alpha emitting radionuclides, including I-131, Y-90, Lu-177, Bi-213, Ac-225, Th-227, or radiotherapy with Ra-223.

103. A method according to claim 101 or 102, wherein the administration is concomitant and/or sequential.
